(19) United States
(12) Patent Application Publication
(10) Pub. No.: US 2026/0258377 A1
(43) Pub. Date: Sep. 3, 2026

(54) NEW FUCOSYLTRANSFERASE FOR IN VIVO SYNTHESIS OF COMPLEX FUCOSYLATED HUMAN MILK OLIGOSACCHARIDES

(71) Applicant: DSM IP Assets B.V., Maastricht (NL)

(72) Inventor: Manos PAPADAKIS, Hørsholm (DK)

(21) Appl. No.: 18/994,171

(22) PCT Filed: Jul. 14, 2023
(86) PCT No.: PCT/EP2023/069729

§ 371 Date: Jan. 14, 2025

(30) Foreign Application Priority Data
Jul. 15, 2022 (DK) PA202200688

(51) Int. Cl. C12N 9/10; C07H 3/06; C12N 15/70; C12P 19/04

(52) U.S. Cl. CPC C12N 9/1051; C07H 3/06; C12N 15/70; C12P 19/04

(57) ABSTRACT

The present invention relates to the production of fucosylated Human Milk Oligosaccharides (HMOs), and in particular to the production of complex fucosylated HMOs with five or more monosaccharide units such as LNFP-II, LNFP-III and LNDFH-I from precursor oligosaccharides and the genetic engineering of suitable cells for use in said production, as well as to methods for producing said fucosylated HMOs.

### FIELD

The present invention relates to the production of complex fucosylated Human Milk Oligosaccharides (HMOs) and in particular to the production of complex fucosylated HMOs with five or more monosaccharide units, such as LNFP-II, LNFP-III and LNDFH-I, as well as to genetically engineered cells suitable for use in said production.

### BACKGROUND

The design and construction of bacterial cell factories to produce fucosylated Human Milk Oligosaccharides (HMOs), especially for producing more complex fucosylated Human Milk Oligosaccharides (HMOs), is of paramount importance to provide innovative and scalable solutions for the more complex products of tomorrow.

Production of complex fucosylated HMOs has e.g., been described in WO2016/040531, suggesting that the alpha1,3-fucosyltransferase CafD can produce LNFP-III, and in WO2019/008133, describing the alpha1,3-fucosyltransferase FucT109 which appears to fucosylate both the glucose (Glc) and N-acetylglucosamine (GlcNAc) moiety of Lacto-N-neotetraose (LNnT), thus potentially generating a mixture containing all three of LNnT, LNFP-III and LNFP-VI.

Dumon et al., 2004 (alpha-1,3-fucosyltransferase Biotechnol. Prog. 2004, 20, 412-419) further describes an alpha1,3-fucosyltransferase, FutB, which is also suggested to produce a mixture of LNnT, LNFP-III, LNFP-VI and LNDFH-III.

The fucosyltransferases disclosed in the prior art (CafD, FucT109 and FutB), however, only produce minor amounts, if any, of the complex fucosylated HMOs, with high by-product formation.

In summary, production of fucosylated HMOs, especially of more complex fucosylated HMOs, may be challenging due to the lack of fucosyltransferases with the desired substrate specificity, as well as low production yields of the desired fucosylated HMOs as compared to other HMO products present after fermentation, such as HMO precursor products, which may require laborious separation procedures.

### SUMMARY OF THE INVENTION

The need for highly substrate specific α-1,3-fucosyltransferases and α-1,4-fucosyltransferases is solved by the identification of the bifunctional α-1,3(4)-fucosyltransferase Med1, which exhibits low or no substrate specificity for the glucose moiety in LNT and LNnT, but which is highly substrate specific for the N-acetylglucosamine (GlcNAc) moiety in LNT and LNnT. This allows Med1 to produce high levels of and very pure LNFP-III in an LNnT background strain without producing any other complex fucosylated HMOs. What is more, the very specific α-1,4-fucosyltransferase activity of Med1 towards the GlcNAc moiety in LNT is rare, and it is herein for the first time shown that an α-1,4-fucosyltransferase can produce the complex fucosylated HMOs LNFP-II and/or LNDFH-I. The HMOs produced by a strain containing the Med1 α-1,3(4)-fucosyltransferase can be mixtures of HMOs that comprise the precursor HMOs, such as LNT, LNnT or LNFP-I, and/or by-product oligosaccharides or by-product HMOs, such as 2′FL, 3FL, DFL, pLNH2 or pLNnH. The mixtures are however essentially free of the alternative complex fucosylated HMOs LNFP-V, LNFP-VI and/or LNDFH-II.

The α-1,3(4)-fucosyltransferase presented herein may therefore be used in the production of pure, or essentially pure LNFP-III and LNFP-II in a mixture of HMOs from which it may be easily purified. In addition, the herein identified α-1,3(4)-fucosyltransferase, for the first time enables an efficient biosynthesis of the di-fucosylated HMO LNDFH-I. Hence, provided herein is an enzyme, mixtures, compositions, uses, genetically engineered cells and methods for the production of LNFP-II, LNFP-III and/or LNDFH-I.

A first aspect described herein therefore relates to a genetically engineered cell, which is capable of producing one or more fucosylated HMOs, comprising a recombinant nucleic acid sequence encoding a fucosyltransferase with α-1,3(4)-fucosyltransferase activity, wherein the glycosyltransferase is Med1 with an amino acid sequence according to SEQ ID NO: 1, or a functional homologue thereof with an amino acid sequence that is at least 80% identical to SEQ ID NO: 1.

A second aspect of the current invention relates to a method for producing one or more fucosylated HMOs, preferably, wherein the HMOs are selected from the group consisting of LNFP-II, LNFP-III and LNDFH-I, said method comprises culturing a genetically engineered cell capable of producing one or more fucosylated HMOs, comprising a recombinant nucleic acid sequence encoding a fucosyltransferase with α-1,3(4)-fucosyltransferase activity, wherein the glycosyltransferase is Med1 with an amino acid sequence according to SEQ ID NO: 1, or a functional homologue thereof with an amino acid sequence that is at least 80% identical to SEQ ID NO: 1.

A third aspect relates to use of an enzyme with α-1,3(4)-fucosyltransferase activity for the production of a fucosylated product, such as a fucosylated HMO, preferably selected from the group consisting of LNFP-II, LNFP-III and LNDFH-I, wherein the enzyme is Med1 with an amino acid sequence according to SEQ ID NO: 1, or a functional homologue thereof with an amino acid sequence that is at least 80% identical to SEQ ID NO: 1.

A fourth aspect relates to a mixture of HMOs consisting essentially of
a) LNFP-II, pLNH2 and LNT, or
b) LNFP-II, LNT and 3FL, or
c) LNFP-II, LNT and pLNH2, or
d) LNFP-II, LNT-II, LNT, 3FL and pLNH2, or
e) LNFP-III and LNnT, or
f) LNFP-III, LNnT and pLNnH, or
g) LNFP-III and 3FL or
h) LNDFH-I, LNFP-I and 2′FL.

A fifth aspect relates to
a) a mixture of HMOs consisting essentially of LNFP-II and LNT, and with low amounts of 3FL or pLNnH, such as below 20% total molar HMO content in the mixture,
b) a mixture of HMOs consisting essentially of 5-35 molar % LNFP-II, 0-15 molar % LNT-II, 0-20 molar % 3FL and 50-85 molar % LNT in total adding up to 100% molar content,
c) a mixture of HMOs consisting essentially of LNFP-III and LNnT, and with low amounts of 3FL or pLNnH, such as below 10% total molar HMO content in the mixture,
d) a mixture of HMOs consisting essentially of 50-99 molar % LNFP-III, 0-45 molar % LNnT, 0-20% 3FL, and 0-10% pLNnH, in total adding up to 100% molar content,
e) a mixture of HMOs is provided consisting essentially of LNDFH-I, LNFP-I and 2′FL, and
f) a mixture of HMOs consisting essentially of 7-15 molar % LNDFH-1, 65-90 molar % LNFP-I and 5-20 molar % 2′FL with the total molar HMO content in the mixture adding up to 100 molar %.

A sixth aspect of the invention relates to compositions comprising and/or essentially consisting of the mixtures of a) to f) and to use thereof in infant formula, a dietary supplement and/or medical nutrition.

### BRIEF DESCRIPTION OF THE FIGURES

FIG. 1 is an illustration of the pathway for making LNFP-II from an LNT background strain and the potential by-products that can be generated in the process. The accepter for the alpha-1,3(4)-fucosyltransferases can be lactose, LNT, LNFP-V and LNFP-II, depending on the specificity of the alpha-1,3(4)-fucosyltransferase. Fucosylated by-products are for example 3FL, LNFP-V and LNDFH-II. Neutral by-products are for example LNT-II, LNnT and pLNH2. Depending on the genetically modified cell the initial substrate for the LNFP-II production may be lactose, LNT-II or even LNnT, as long as the strain can take up the initial substrate. In the figure β1,3-GlcNAcT is a β1,3-glucosaminyltransferase, β1,3-GalT is a β1,3-galactosyl-transferase, Glc α1,3(4)-FucT is a α1,3(4)-fucosyltransferase with activity on glucose moieties in the acceptor oligosaccharide, GlcNAc α1,3(4)-FucT is a α1,3(4)-fucosyltransferase with activity on the GlcNAc moiety in the acceptor oligosaccharide; GDP is guanosine-diphosphate and UDP is uridine-diphosphate.

FIG. 2 is an illustration of the pathway for making LNDFH-I from an LNFP-I background strain and the potential by-products that can be generated in the process. The accepter for the alpha-1,3(4)-fucosyltransferases can be lactose, LNT, LNFP-I and LNFP-II, depending on the specificity of the alpha-1,3(4)-fucosyltransferase. Fucosylated by-products are for example 2′FL, 3FL, LNFP-I, and LNFP-II. Neutral by-products are for example LNT-II, LNnT and pLNH2. Depending on the genetically modified cell the initial substrate for the LNDFH-I production may be lactose, LNT-II or even LNnT or LNFP-I, as long as the strain can take up the initial substrate. In the figure β1,3-GlcNAcT is a β1,3-glucosaminyltransferase, β1,3-GalT is a β1,3-galactosyltransferase, α1,2-FucT is a α1,2-fucosyltransferase, α1,3(4)-FucT is a α1,3(4)-fucosyltransferase; GDP is guanosine-diphosphate and UDP is uridine-diphosphate.

FIG. 3 is an illustration of the pathway for making LNFP-III from an LNT background strain and the potential by-products that can be generated in the process. The accepter for the alpha-1,3-fucosyltransferases can be lactose, LNnT, LNFP-VI and LNFP-III, depending on the specificity of the alpha-1,3-fucosyltransferase. Fucosylated by-products are for example 3FL, LNFP-VI, and LNDFH-III. Neutral by-products are for example LNT-II, LNnT and pLNnH. Depending on the genetically modified cell the initial substrate for the LNFP-III production may be lactose, LNT-II or even LNnT, as long as the strain can take up the initial substrate. In the figure β1,3-GlcNAcT is a β1,3-glucosaminyltransferase, β1,3-GalT is a β1,3-galactosyltransferase, Glc α1,3-FucT is a α1,3-fucosyltransferase with activity on glucose moieties in the acceptor oligosaccharide, GlcNAc α1,3-FucT is a α1,3-fucosyltransferase with activity on the GlcNAc moiety in the acceptor oligosaccharide; GDP is guanosine-diphosphate and UDP is uridine-diphosphate.

FIG. 4: Shows the experimental setup of the regeneration and viability assessment of lyophilized probiotics under pH 3.0 acidic conditions.

FIG. 5: Shows the regeneration and viability of lyophilized Lactobacillus casei (DSM 32382), incubated for 3 h at pH 3.0. A) is the control without HMOs, the picture from left to right show dilution steps 1:100 (E-2), 1:1000 (E-3). B) is Lactobacillus casei (DSM 32382) in combination with an HMO mixture containing 75% LNFP-1, 10% LNDFH-I and 15% 2′FL, the picture from left to right show dilution steps 1:100 (E-2), 1:1000 (E-3), 1:10.000 (E-4) and 1:100.000 (E-5). C) shows the results expressed as mean values (n=2) with standard deviation (SD) of colony-forming units (CFU) per milliliter calculated from Lactobacillus casei colonies on agar plates when plated at dilution step E-3, ** indicates statistically significant difference relative to control, p<0.01.

### DETAILED DESCRIPTION

The present invention approaches the biotechnological challenges of in vivo HMO production of, in particular, complex fucosylated HMOs which comprise at least five monosaccharide units, of which at least one monosaccharide unit is a fucosyl unit. Preferably, the complex fucosylated HMOs are selected from the group consisting of the complex fucosylated HMOs LNFP-II, LNFP-III and LNDFH-I. The present invention offers specific strain engineering solutions to produce specific complex fucosylated HMOs, in particular, LNFP-II, LNFP-III and/or LNDFH-I, by exploiting the substrate specificity of the α-1,3(4)-fucosyltransferase, Med1, disclosed herein towards the GlcNAc moiety in LNT and LNnT and the dual activity of the α-1,3(4)-fucosyltransferase, Med1, of the present disclosure.

A genetically engineered cell of the present invention expresses genes encoding key enzymes for the biosynthesis of fucosylated HMOs. In addition, it is advantageous if the genetically engineered cell expresses the genes needed to produce LNT, LNnT or LNFP-I, either from lactose or LNT-II as the initial substrate. In some embodiments a genetically engineered cell of the present invention further expresses the de novo GDP-fucose pathway genes responsible for the formation of GDP-fucose manA, manB, manC, gmd and wcaG. It may be advantageous to overexpress one or more of these genes and/or to upregulate the colanic acid gene cluster (CA), including the genes gmd, wcaG, wcaH, wcal, manC and manB from E. Coli, through introduction of a nucleic acid construct encoding the CA as shown in SEQ ID NO: 3, allowing for formation of GDP-fucose, which enables the cell to produce a higher level of fucosylated oligosaccharide from one or more oligosaccharide substrates, such as lactose or LNT-II, LNT, LNnT and/or LNFP-I. Depending on the intended use of substrate, one or more additional glycosyltransferases and pathways for producing nucleotide-activated sugars, such as glucose-UDP-GlcNac, CMP-N-acetylneuraminic acid, UDP-galactose, UDP-glucose, UDP-N-acetylglucosamine, UDP-N-acetylgalactosamine and/or CMP-N-acetylneuraminic acid can also be present in the genetically engineered cell.

### Production of LNFP-II, LNFP-III or LNDFH-I

The advantage of using the α-1,3(4)-fucosyltransferase of the present disclosure is its ability to specifically recognize and fucosylate the GlcNAc moiety in LNT, LNnT and/or LNFP-I, to generate LNFP-II, LNFP-III and/or LNDFH-I, respectively (as indicated by the reactions in formula 1, 2 and 3). In essence, the present disclosure for the first time describes an enzyme with dual α-1,3-fucosyltransferase and 1,4-fucosyltransferase activity (α-1,3(4)-fucosyltransferase) that is more active on the GlcNAc moiety in LNnT, than e.g., the pure α-1,3-fucosyltransferases FucT109 (WO2019/008133) and CafD (WO2016/040531), which have prior been suggested to fucosylate both GlcNAc and Glucose (Glu) moieties in the α-1,3 position on LNnT.

Furthermore, the α-1,3(4)-fucosyltransferase Med1 of the present invention is also capable of fucosylating the GlcNAc moiety in LNT, with high specificity, in an alpha-1,4 linkage to produce LNFP-II with essentially no production of LNFP-V. This is in contrast to FucT109, which has prior been suggested to also fucosylate the Glc moiety of LNT, but only in an alpha-1,3 linkage, thus producing LNFP-V.

The α-1,3(4)-fucosyltransferase described herein, med1, has very low or no activity on glucose and galactose moieties in an oligosaccharide, in particular med1 has low or no activity on the glucose and galactose moieties in an oligosaccharide with an LNT or LNnT backbone. Essentially this means that the med1 enzyme is capable of producing HMO mixtures where there only is one complex fucosylated oligosaccharide present in the mixture.

Furthermore, the α-1,3(4)-fucosyltransferase described herein has very low activity on the glucose moiety in lactose as well as in LNT and LNnT. Thus, if LNT or LNnT is available in sufficient amounts inside the genetically engineered cell, very little 3FL, if any, is produced by the α-1,3(4)-fucosyltransferase described in the present disclosure.

The traits of the α-1,3(4)-fucosyltransferase described herein are therefore well-suited for high-level industrial production of LNFP-III without production of high levels of alternatively fucosylated HMOs (side products), such as LNFP-VI and LNDFH-III and other by-product HMOs such as LNnT and pLNnH. Furthermore, the α-1,3(4)-fucosyltransferase described herein is also well-suited for producing LNFP-II and mixtures comprising LNFP-II and LNT and minor amounts of LNT-II, 3FL and pLNH2. Additionally, the α-1,3(4)-fucosyltransferase disclosed herein is also suited for producing LNDFH-I and mixtures comprising LNDFH-I and LNFP-I, due to its ability to further fucosylate LNFP-I to produce LNDFH-I.

The genetically engineered cells of the present disclosure, which express the α-1,3(4)-fucosyltransferase, Med1, with high substrate specificity for the GlcNAc moiety in LNnT, LNT and LNFP-I, for the first time enable the production of high titters of LNFP-III and mixtures of HMOs containing LNPF-II or LNDFH-I, from which LNPF-II or LNDFH-I can potentially be purified. In particular the HMO mixtures produced by the genetically engineered cells of the present disclosure, which express the α-1,3(4)-fucosyltransferase, Med1, only produce a single complex fucosylated HMO consisting of at least five monosaccharide units, which poses a large advantage if the complex HMO, such as LNFP-II, LNFP-III or LNDFH-II is to be purified from the mixture.

Thereby, the present disclosure enables a more efficient LNFP-II, LNFP-III and/or LNDFH-I production, which is highly beneficial in biotechnological production of more complex fucosylated HMOs, such as LNFP-II, LNFP-III and LNDFH-I.

In the following sections, individual elements of the invention, and in particular of the genetically engineered cell are described. It is understood that these elements can be combined across the individual sections.

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). Preferably, the oligosaccharide is an HMO.

### Human Milk Oligosaccharide (HMO)

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an alpha-L-fucopyranosyl and/or an alpha-N-acetyl-neuraminyl (fucosyl) moiety. HMO structures are e.g., disclosed by Xi Chen in Chapter 4 of Advances in Carbohydrate Chemistry and Biochemistry 2015 vol 72.

The present invention focuses on fucosylated HMO's. Examples of fucosylated HMOs include, 2′-fucosyllactose (2′FL), lacto-N-fucopentaose I (LNFP-I), lacto-N-difucohexaose I (LNDFH-I), 3-fucosyllactose (3FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose VI (LNFP-VI), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II), fucosyl-lacto-N-neohexaose (FLNnH), 3-fucosyl-3′-fucosyllactose (FSL), fucosyl-LST-a (FLST-a), fucosyl-LST b (FLST b), fucosyl-LST-c (FLST-c), fucosyl-LST d (FLST-d) and fucosyl-lacto-N-hexaose (SLNH).

In the context of the present invention, complex fucosylated HMOs are fucosylated HMOs that comprises at least 5 monosaccharide units of which at least one monosaccharide unit is a fucosyl unit, non-limiting examples of complex fucosylated HMOs are the fucosylated HMOs consisting of 5 monosaccharide units e.g., LNFP-I, LNFP-II, LNFP-III, LNFP-V and LNFP-VI and complex fucosylated HMO with 6 monosaccharide units such as but not limited to LNDFH-I, LNDFH-II and LNDFH-III. Preferably, a complex fucosylated HMO is one that require at least three different glycosyltransferase activities to be produced from lactose as the initial substrate, e.g., the formation of LNFP-II requires an α-1,4-fucosyltransferase, a β-1,3-N-acetyl-glucosaminyl-transferase and a β-1,3-galactosyltransferase, the formation of LNFP-III requires an α-1,3-fucosyltransferase, a β-1,3-N-acetyl-glucosaminyl-transferase and a β-1,4-galactosyltransferase, and the formation of LNDFH-I requires an α-1,2-fucosyltransferase, an α-1,4-fucosyltransferase, a β-1,3-N-acetyl-glucosaminyl-transferase and a β-1,4-galactosyltransferase.

In particular, the fucosylated HMO(s) produced is/are selected from complex fucosylated HMOs comprising at least five monosaccharide units of which at least one monosaccharide unit is a fucosyl unit. Moreover, in embodiments the fucosylated HMOs is/are selected from complex fucosylated HMOs with an LNT and/or LNnT backbone structure, preferably, selected from the group consisting of LNFP-II, LNFP-III and LNDFH-I.

Examples of fucosylated HMOs with an LNT backbone structure are lacto-N-fucopentaose I (LNFP-I), lacto-N-fucopentaose II (LNFP-II), lacto-N-fucopentaose V (LNFP-V), Lacto-N-difucohexaose I (LNDFH-I), Lacto-N-difucohexaose II (LNDFH-II), sialyl-lacto-N-fucopentaose I (S-LNFP-I), sialyl-lacto-N-fucopentaose II (S-LNFP-II), Mono-Fucosyl-lacto-N-hexaose I (F-LNH-I), Mono-Fucosyl-lacto-N-hexaose II (F-LNH-II), Mono-fucosyl-lacto-N-hexaose III (F-LNH-III), Difucosyl-lacto-N-hexaose I (DF-LNH-I), Difucosyl-lacto-N-hexaose II (DF-LNH-II), Difucosyl-Lacto-N-hexaose III (DF-LNH-III), Trifucosyl-lacto-N-hexaose (TF-LNH), Fucosyl-sialyl-lacto-N-hexaose I (FS-LNH) and Disialyl-fucosyl-lacto-N-hexaose II (DS-F-LNH-II).

Examples of HMOs with an LNnT backbone structure are Lacto-N-fucopentaose III (LNFP-III), Lacto-N-fucopentaose VI (LNFP-VI), Lacto-N-difucohexaose III (LNDFH-III), Fucosyl-para-lacto-N-hexaose I (F-para-LNH-I), difucosyl-para-lacto-N-hexaose (DF-para-LNH), difucosyl-para-lacto-N-neohexaose (DF-para-LNnH) and fucosyl-sialyl-lacto-N-neohexaose I (FS-LNnH-I).

In embodiments of the present disclosure, the fucosyltransferase of the present invention predominantly fucosylates the N-acetylglucoseamine (GlcNAc) moiety of LNT, LNnT or LNFP-I. In further embodiments, the fucosyltransferase of the present invention only fucosylates the N-acetylglucoseamine (GlcNAc) moiety of LNT, LNnT or LNFP-I. In that regard, the one or more fucosylated HMOs is/are selected from the group consisting of LNFP-II, LNFP-III and LNDFH-I.

### An Acceptor Oligosaccharide

A genetically engineered cell according to the present invention comprises a recombinant nucleic acid sequence encoding a fucosyltransferase with α-1,3(4)-fucosyltransferase activity capable of transferring fucose from an activated sugar to the GlcNAc moiety of an acceptor oligosaccharide, in an α-1,3 linkage or α-1,4 linkage.

In the context of the present invention, an acceptor oligosaccharide is an oligosaccharide that can act as a substrate for a glycosyltransferase capable of transferring a glycosyl moiety from a glycosyl donor to the acceptor oligosaccharide. The glycosyl donor is preferably a nucleotide-activated sugar as described in the section on “glycosyltransferases”. Preferably, the acceptor oligosaccharide is a precursor for making a more complex HMO and can also be termed the precursor molecule.

The acceptor oligosaccharide can be either an intermediate product of the present fermentation process, an end-product of a separate fermentation process employing a separate genetically engineered cell, or an enzymatically or chemically produced molecule.

In the present context, said acceptor oligosaccharide for the α-1,3(4)-fucosyltransferase is preferably an oligosaccharide with a lacto-N-tetraose (LNT) or lacto-N-neotetraose (LNnT) backbone. Preferably the acceptor oligosaccharide for the α-1,3(4)-fucosyltransferase is lacto-N-tetraose (LNT) or lacto-N-neotetraose (LNnT). The LNT and LNnT can be produced inside the genetically engineered from the precursor molecules lactose (e.g., acceptor for the β-1,3-N-acetyl-glucosaminyl-transferase) and/or lacto-N-triose II (LNT-II) (e.g., acceptor for the β-1,4-galactosyltransferase or β-1,3-galactosyltransferase). In addition the acceptor oligosaccharide for the α-1,3(4)-fucosyltransferase may also be lacto-N-fucopentaose I (LNFP-I) which is produced from the precursor molecules lactose (e.g., acceptor for the β-1,3-N-acetyl-glucosaminyl-transferase) and/or lacto-N-triose II (LNT-II) (e.g., acceptor for the β-1,3-galactosyltransferase) and/or LNT (e.g., acceptor for the α-1,2-fucosyltransferase). The precursor molecule is preferably fed to the genetically engineered cell, which is capable of producing LNT, LNnT or LNFP-I from the precursor.

### Glycosyltransferases

The genetically engineered cell according to the present invention comprises at least one recombinant nucleic acid sequence encoding at least one glycosyltransferase capable of transferring a fucosyl residue from a fucosyl donor to an acceptor oligosaccharide to synthesize one or more fucosylated human milk oligosaccharide product, i.e., a fucosyltransferase.

The genetically engineered cell according to the present invention may comprise one or more further recombinant nucleic acids encoding one or more heterologous glycosyltransferases capable of transferring a glycosyl residue from a glycosyl donor to an acceptor oligosaccharide. Preferably, the additional glycosyltransferase(s) enables the genetically engineered cell to synthesize LNT, LNnT of LNFP-I from a precursor molecule, such as lactose or LNT-II, or LNT (precursor for LNFP-I). In embodiments, the genetically engineered cell of the present invention, comprises one or more further recombinant nucleic acids encoding one or more heterologous glycosyltransferases.

The additional glycosyltransferase is preferably selected from the group consisting of, galactosyltransferases, glucosaminyltransferases, fucosyltransferases and N-acetylglucosaminyl transferases.

In one aspect, the fucosyltransferase in the genetically engineered cell of the present invention is an α-1,3(4)-fucosyltransferase. Preferably, the α-1,3(4)-fucosyltransferase is capable of transferring a fucose unit onto the GlcNAc moiety of an LNT, LNnT or LNFP-I molecule.

In the present invention, the functional enzyme (α-1,3(4)-fucosyltransferase) capable of transferring a fucosyl moiety from a fucosyl donor to an acceptor oligosaccharide is Med1 with an amino acid sequence according to SEQ ID NO: 1, or a functional homologue thereof with an amino acid sequence that is at least 80% identical to SEQ ID NO: 1. This enzyme can e.g., be used to produce LNFP-II, LNFP-III and/or LNDFH-III.

Without being bound by theory, an α-1,3(4)-fucosyltransferase with a higher substrate specificity for the GlcNAc moiety in LNT, LNnT and LNFP-I compared to the substrate specificity for the terminal galactose moiety in LNT, LNnT or LNFP-I is be advantageous as such an α-1,3(4)-fucosyltransferase would in theory produce less or no alternatively complex fucosylated HMOs, such as LNFP-V and LNFP-VI, when the initial substrate is lactose and wherein the availability of LNnT is in such a case not limited. A lower amount of alternatively complex fucosylated HMOs would in such a case result in an easier purification of the produced HMOs, as the purification of LNFP-II, LNFP-III or LNDFH-I from a mixture of HMOs predominantly comprising the intended product would be simpler, as it is easier to separate the complex fucosylated HMOs from smaller HMOs than separating different fucosylated HMOs of the same or similar size from each other, e.g., LNFP-III from LNFP-VI or LNDFH-III, or LNFP-II from LNFP-V or LNDFH-II, or LNDFH-I from LNDFH-II. Hence a lower initial amount LNFP-VI and/or LNDFH-III in LNFP-III production is considered beneficial in the purification of LNFP-III, while a lower initial amount LNFP-V and/or LNDFH-II in LNFP-II production is considered beneficial in the purification of LNFP-II, and a lower initial amount LNDFH-II in LNDFH-I production is considered beneficial in the purification of LNDFH-I.

In preferred embodiments, the use of an α-1,3(4)-fucosyltransferase according to the present invention results in that at least 80% of the molar content of the total HMOs produced by a cell according to the present invention is LNFP-III. In other preferred embodiments, the use of an α-1,3(4)-fucosyltransferase according to the present invention results in that at least 15% of the molar content of the total HMOs produced by a cell according to the present invention is LNFP-II. In other preferred embodiments, the use of an α-1,3(4)-fucosyltransferase according to the present invention results in that at least 8% of the molar content of the total HMOs produced by a cell according to the present invention is LNDFH-I. IN embodiment, the α-1,3(4)-fucosyltransferase is Med1, with an amino acid sequence according to SEQ ID NO: 1 or a functional homologue thereof with an amino acid sequence that is at least 80% identical to SEQ ID NO: 1.

In embodiments, the expression of an α-1,3(4)-fucosyltransferase according to the present invention in a genetically engineered cell is further combined with expression of one or more further recombinant nucleic acids encoding one or more heterologous glycosyltransferases.

In embodiments of the present invention the cell further comprises one or more recombinant nucleic acid sequence encoding a β-1,3-galactosyltransferase and/or a β-1,4-galactosyltransferase.

In preferred embodiments, the expression of an α-1,3(4)-fucosyltransferase of the invention in a genetically engineered cell is combined with expression of a β-1,3-galactosyltransferase such as galTK from Helicobacter pylori or of a β-1,4-galactosyltransferase, such as galT from Helicobacter pylori. In a further embodiment, a third enzyme is expressed, such as a β-1,3-N-acetyl-glucosaminyl-transferase, e.g., LgtA from Neisseria meningitidis. In a yet further embodiment, a fourth enzyme is expressed, such as a α-1,2-fucosyltransferase, e.g., Smob from Sulfuriflexus mobilis.

Exemplified further glycosyltransferases in addition to the α-1,3(4)-fucosyltransferase Med1, are preferably selected from the glycosyltransferases described below (tables 1, 2 and 3).

α-1,3(4)-fucosyltransferase

The term “α-1,3(4)-fucosyltransferase” refers to a glycosyltransferase that catalyzes the transfer of fucosyl from a donor substrate, such as GDP-fucose, to an acceptor molecule in an α-1,3-linkage. Preferably, an α-1,3(4)-fucosyltransferase used in the present invention does not originate in the species of the genetically engineered cell, i.e., the gene encoding the α-1,3(4)-fucosyltransferase is of heterologous origin and is Med1 from Mediterranea sp. An20 with an amino acid sequence according to SEQ ID NO: 1 and with the GenBank ID WP_087337236.1, or a functional homologue thereof with an amino acid sequence that is at least 80% identical to SEQ ID NO: 1. In the context of the present invention, the acceptor molecule for the α-1,3(4)-fucosyltransferase is preferably the GlcNAc moiety of an acceptor oligosaccharide of at least four monosaccharide units, e.g., LNT, LNnT or LNFP-I.

Exemplified reaction for production of LNFP-II from LNT using an α-1,3(4)-fucosyltransferase of the present invention is illustrated in formula 1:

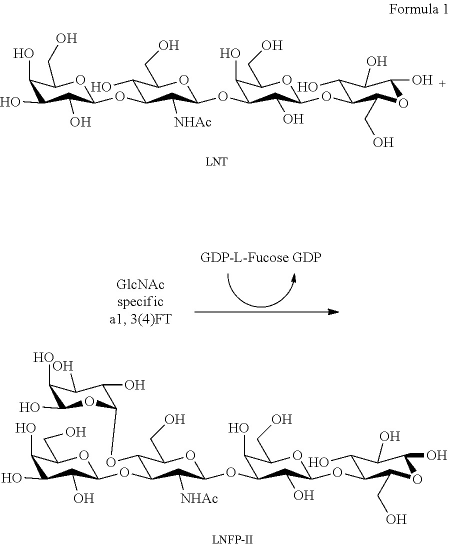

Exemplified reaction for production of LNFP-III from LNnT using an α-1,3(4)-fucosyltransferase of the present invention is illustrated in formula 2:

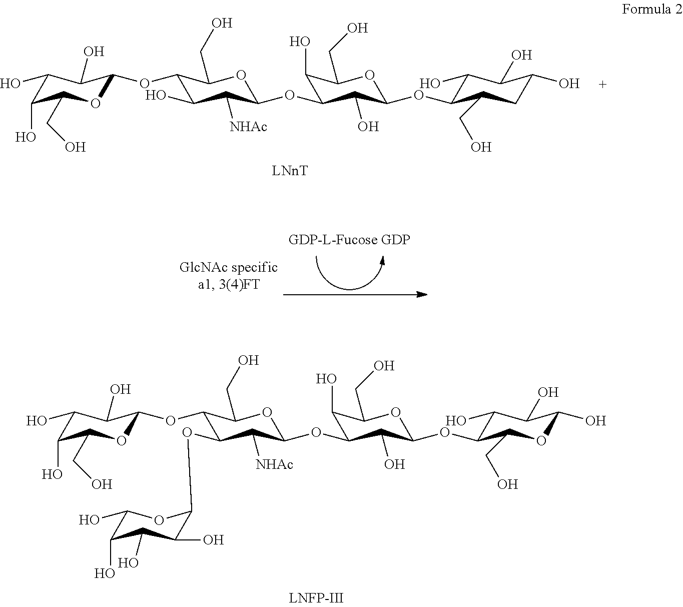

Exemplified reaction for production of LNDFH-I from LNFP-I using an α-1,3(4)-fucosyltransferase of the present invention is illustrated in formula 3:

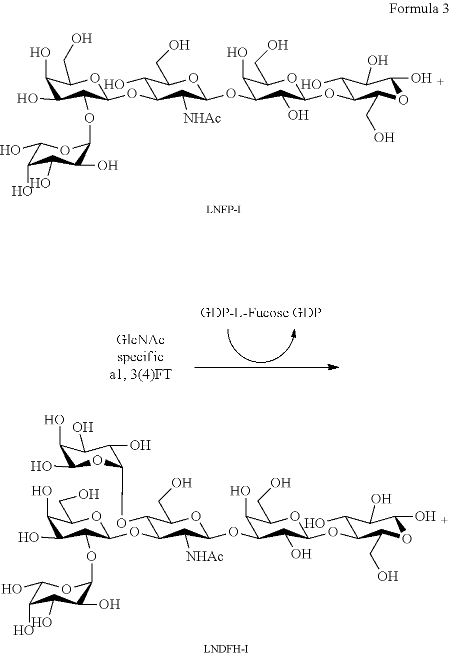

The α-1,3(4)-fucosyltransferase can be selected from a functional homologue with an amino acid sequence with at least 80%, such as at least 85%, such as at least 90%, such as at least 95%, or such as at least 99% sequence identity, such as 100% identical to the amino acid sequence of any one of the α-1,3(4)-fucosyltransferase of SEQ ID NO: 1. In preferred embodiments the functional homologue has a tryptophan (w) in the position corresponding to position 13 of SEQ ID NO: 1 when aligned according to the sequence identity assessment parameter and tools described herein.

Example 1 of the present application discloses the identification of the fucosyltransferases Para1 (SEQ ID NO: 33) and Med1 (SEQ ID NO: 1), which were both capable of producing LNFP-II. There was, however, a distinct difference between the substrate specificity of the Para1 and Med1 fucosyltransferases in that Med1 exclusively fucosylated LNT at the GlcNAc moiety with an α-1,4 linkage, thus only producing a single fucosylated HMO with an LNT backbone, specifically LNFP-II, whereas Para1 fucosylated LNT at the glucose (Glc) moiety and at the GlcNAc moiety, thus producing three fucosylated HMOs with an LNT backbone, LNFP-II, LNFP-V and LNDFH-II, respectively. Since 50 fucosyltransferase enzymes were screened in total and only two were identified as being capable of producing LNFP-II, and only Med1 was capable of producing LNFP-II as the sole complex fucosylated HMO, the LNT GlcNAc specificity appears to be rare among fucosyltransferases across bacterial species.

Furthermore, example 1 of the present invention discloses that increasing the copy number of Med1 to two genetic copies increased the LNFP-II production, whereas increasing the copy number of Para1 only resulted in a minor increase in LNFP-II formation, whereas the amount of the difucosylated HMO LNDFH-II was increased to a greater extent, clearly indicating that Para1 has equal preference for fucosylating the GlcNAc and the Glc moiety of LNT, whereas Med1 only possess activity towards the GlcNAc moiety. Further increasing the Med1 expression was investigated by introduction of the high copy plasmid pUC57-Med1-PglpF (Med1_3) containing the Med1 encoding sequence which increased the LNFP-II level further. Similar effects were observed for production of LNFP-III (example 2) and LNDFH-I (examples 3 and 5).

The absence of alternative fucosylated LNT or LNnT species when using Med1 is highly advantageous and preferred if it is desired to purify LNFP-II, LNFP-III or LNDFH-I from a broth which does not contain additional fucosylated HMOs of similar size such as LNFP-V, LNFP-VI, or LNDFH-II. Thus, when the aim is to produce high amounts of a single HMO, a high level of that specific HMO is beneficial.

In embodiments of the invention, the fucosyltransferase with α-1,3(4)-fucosyltransferase activity is Med1 from Mediterranea sp. An20 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1.

The enzyme Med1 is in particular, introduced into a genetically engineered cell which further comprises a β-1,3-galactosyltransferase or a β-1,4-galactosyltransferase and preferably also a β-1,3-N-acetyl-glucosaminyl-transferase.

β-1,3-N-acetyl-glucosaminyl-transferase

A β-1,3-N-acetyl-glucosaminyl-transferase is any protein which comprises the ability of transferring the N-acetyl-glucosamine of UDP-N-acetyl-glucosamine to lactose or another acceptor molecule, in a β-1,3-linkage. Preferably the β-1,3-N-acetyl-glucosaminyl-transferase used herein does not originate in the species of the genetically engineered cell, i.e., the gene encoding the β-1,3-N-acetyl-glucosaminyl-transferase is of heterologous origin. In the context of the present invention, the acceptor molecule is either lactose or an oligosaccharide of at least four monosaccharide units, e.g., LNT, LNnT, or more complex HMO structures. Accordingly, in embodiments, the genetically engineered cell further comprises one or more recombinant nucleic acid sequence(s) encoding a β-1,3-N-acetyl-glucosaminyltransferase.

Non-limiting examples of β-1,3-N-acetyl-glucosaminyltransferases are given in table 1. β-1,3-N-acetyl-glucosaminyltransferase variants may also be useful, preferably such variants are at least 80%, such as at least 85%, such as at least 90, such as at least 95% identical to the amino acid sequence of any one of the β-1,3-N-acetyl-glucosaminyltransferase in table 1.

TABLE 1
List of β-1,3-N-acetyl-glucosaminyltransferase

| Protein Name | GenBank ID | origin |
|---|---|---|
| lgtA | WP_033911473.1 | Neisseria meningitidis |
| | WP_002248149 | |
| | AAF42258.1 | |
| | ELK60643.1 | |
| lgtA | AAK70338.1 | Neisseria gonorrhoeae |
| | ACF31229.1 | |
| lgtA | AAK02595.1 | Pasteurella multocida |
| lgtA | EEZ72046.1 | Neisseria cinerea |
| HD0466 | WP_010944479.1 | Haemophilus ducreyi |
| PmnagT | WP_014390683.1 | Pasteurella multocida |

In embodiments, the genetically engineered cell comprises a recombinant nucleic acid sequence encoding a β-1,3-N-acetyl-glucosaminyltransferase. In one embodiment, the recombinant nucleic acid sequence encoding a β-1,3-N-acetylglucosaminyltransferase comprises or consists of the amino acid sequence of SEQ ID NO: 4 (LgtA from N. meningitidis) or a functional homologue thereof with an amino acid sequence with at least 80%, such as at least 85%, such as at least 90%, such as at least 95%, or such as at least 99% sequence identity to SEQ ID NO: 4.

For the production of LNnT or LNT from lactose as substrate, the LNT-II precursor is formed using a β-1,3-N-acetylglucosaminyltransferase. In one embodiment the genetically engineered cell comprises a β-1,3-N-acetylglucosaminyltransferase gene, or a functional homologue or fragment thereof, to produce the intermediate LNT-II from lactose.

Some of the examples below use the heterologous β-1,3-N-acetyl-glucosaminyl-transferase named LgtA from Neisseria meningitidis or a variant thereof.

### β-1,3-Galactosyltransferase

A β-1,3-Galactosyltransferase is any protein that comprises the ability of transferring the galactose of UDP-Galactose to a N-acetyl-glucosaminyl moiety to an acceptor molecule in a beta-1,3-linkage. Preferably, a β-1,3-galactosyltransferase used herein does not originate in the species of the genetically engineered cell i.e., the gene encoding the β-1,3-galactosyltransferase is of heterologous origin.

Non-limiting examples of β-1,3-galactosyltransferases are given in table 12. β-1,3-galactosyltransferases variants may also be useful, preferably such variants are at least 80%, such as at least 85%, such as at least 90, such as at least 95% identical to one of the β-1,3-galactosyltransferases in table 12.

TABLE 12
List of beta-1,3-glycosyltransferases

| Protein Name | GenBank ID | Origin |
|---|---|---|
| WbgO | WP_000582563.1 | E. coli 055:H7 |
| GalTK | WP_111735921.1 | Helicobacter pylori ATCC 43504 |
| CvB3GalT | WP_080969100.1 | Chromobacterium violaceum |

In the context of the present invention the acceptor molecule, is an acceptor saccharide, e.g., LNT-II, or more complex HMO structures.

The examples below use the heterologous β-1,3-galactosyltransferase named GalTK or a variant thereof, to produce e.g., LNFP-II, LNDFH-I or LNFP-I and LNDFH-I in combination with other glycosyl transferases.

In embodiments the cell of the present invention further comprises a recombinant nucleic acid sequence encoding a β-1,3-N-acetyl-glucosaminyltransferase. In one embodiment, the recombinant nucleic acid sequence encoding a β-1,3-galactosyltransferases comprises or consists of the amino acid sequence of SEQ ID NO: 31 (galTK from H. pylori) or a functional homologue thereof with an amino acid sequence with at least 80%, such as at least 85%, such as at least 90%, such as at least 95%, or such as at least 99% identity to SEQ ID NO: 31.

To produce LNT form an LNT-II precursor, a β-1,3-galactosyltransferase is needed. In one embodiment, the genetically modified cell comprises a β-1,3-galactosyltransferase gene, or a functional homologue or fragment thereof.

Below are examples of genetically modified strains according to the present invention with specific combinations of glycosyl transferases that will lead to production of LNFP-II or LNDFH-I using lactose as initial substrate.

In one example, LgtA from Neisseria meningitidis is used in combination with galTK from Helicobacter pylori and Med1 from Mediterranea sp. An20 to produce LNFP-II starting from lactose as initial substrate.

In another example, LgtA from Neisseria meningitidis is used in combination with galTK from Helicobacter pylori, Smob from Sulfuriflexus mobilis and Med1 from Mediterranea sp. An20 to produce LNDFH-I starting from lactose as initial substrate.

In yet another example, galTK from Helicobacter pylori is used in combination with Med1 from Mediterranea sp. An20 to produce LNFP-II starting from LNT-II as initial substrate.

In yet another example, galTK from Helicobacter pylori and Smob from Sulfuriflexus mobilis is used in combination with Med1 from Mediterranea sp. An20 to produce LNFP-II starting from LNT-II as initial substrate.

### β-1,4-Galactosyltransferase

A β-1,4-galactosyltransferase is any protein that comprises the ability of transferring the galactose of UDP-Galactose to a N-acetyl-glucosaminyl moiety to an acceptor molecule in a β-1,4-linkage. Preferably, a β-1,4-galactosyltransferase used herein does not originate in the species of the genetically engineered cell i.e., the gene encoding the β-1,4-galactosyltransferase is of heterologous origin. In the context of the present invention the acceptor molecule, is an acceptor saccharide, e.g., LNT-II, or more complex HMO structures.

The examples below use the heterologous β-1,4-galactosyltransferase named GalT or a variant thereof, to produce e.g., LNFP-III, in combination with other glycosyl transferases. Accordingly, in embodiments, the genetically engineered cell comprises one or more recombinant nucleic acid sequence(s) encoding a β-1,4-galactosyltransferase.

Non-limiting examples of β-1,4-galactosyltransferases are provided in table 2. β-1,4-galactosyltransferases variants may also be useful, preferably such variants are at least 80%, such as at least 85%, such as at least 90, such as at least 95% identical to the amino acid sequence of any one of the β-1,4-galactosyltransferases in table 2.

TABLE 2
List of β-1,4-glycosyltransferases

| Protein Name | GenBank ID | Origin |
|---|---|---|
| GalT | WP_001262061.1 | Helicobacter pylori |
| LgtB | AAF42257.1 | Neisseria meningitidis MC58 |

In embodiments of the present invention the β-1,3-N-acetylglucosaminyltransferase is from Neisseria meningitidis, and the β-1,3-galactosyltransferase and/or β-1,4-galactosyltransferase is from Helicobacter pylori from Helicobacter pylori, respectively.

In one embodiment, the recombinant nucleic acid sequence encoding a β-1,4-galactosyltransferases comprises or consists of the amino acid sequence of SEQ ID NO: 5 (galT from H. pylori) or a functional homologue thereof with an amino acid sequence with at least 80%, such as at least 85%, such as at least 90%, such as at least 95%, or such as at least 99% sequence identity to SEQ ID NO: 5.

To produce LNnT form an LNT-II precursor, a β-1,4-galactosyltransferase is needed. In one embodiment, the genetically engineered cell comprises a β-1,4-galactosyltransferase gene, or a functional homologue or fragment thereof. In embodiments, the β-1,3-N-acetylglucosaminyltransferase is from Neisseria meningitidis and the β-1,4-galactosyltransferase is from Helicobacter pylori. In further embodiments, the 1,3-N-acetylglucosaminyltransferase has an amino acid sequence according to SEQ ID NO: 4, or a functional homologue thereof with an amino acid sequence that is at least 80% identical to SEQ ID NO: 4 and the β-1,4-galactosyltransferase has an amino acid sequence according to SEQ ID NO: 5, or a functional homologue thereof with an amino acid sequence that is at least 80% identical to SEQ ID NO: 5.

To produce LNT form an LNT-II precursor, a β-1,3-galactosyltransferase is needed. In one embodiment, the genetically engineered cell comprises a β-1,3-galactosyltransferase gene, or a functional homologue or fragment thereof. In embodiments, the β-1,3-N-acetylglucosaminyltransferase is from Neisseria meningitidis and the β-1,3-galactosyltransferase is from Helicobacter pylori. In further embodiments, the 1,3-N-acetylglucosaminyltransferase has an amino acid sequence according to SEQ ID NO: 4, or a functional homologue thereof with an amino acid sequence that is at least 80% identical to SEQ ID NO: 4 and the β-1,3-galactosyltransferase is has an amino acid sequence according to SEQ ID NO: 31, or a functional homologue thereof with an amino acid sequence that is at least 80% identical to SEQ ID NO: 31.

Below are non-limiting examples of genetically modified strains according to the present invention with specific combinations of glycosyl transferases that will lead to production of LNFP-II or LNFP-III using lactose as initial substrate.

In a non-limiting example, LgtA from Neisseria meningitidis is used in combination with galT or galTK from Helicobacter pylori and Med1 from Mediterranea sp. An20 to produce LNFP-III or LNFP-II respectively, starting from lactose as initial substrate.

In a non-limiting example, galT or galTK from Helicobacter pylori is used in combination with Med1 from Mediterranea sp. An20 to produce LNFP-III or LNFP-II respectively, starting from LNT-II as initial substrate.

### α-1,2-Fucosyltransferase

An α-1,2-fucosyltransferase is a protein that comprises the ability to catalyze the transfer of fucose from a donor substrate, for example, GDP-fucose, to an acceptor molecule in an α-1,2-linkage. Preferably, an α-1, 2-fucosyltransferase used herein does not originate in the species of the genetically engineered cell i.e., the gene encoding the α-1,2-fucosyltransferase is of heterologous origin. Non-limiting examples of α-1,2-fucosyltransferase are given in table 3. A-1,2-fucosyltransferase variants may also be useful, preferably such variants are at least 80%, such as at least 85%, such as at least 90, such as at least 95% identical to one of the α-1,2-fucosyltransferase in table 3.

TABLE 3
List of α-1,2-fucosyltransferase

| Protein Name | GenBank ID | Origin |
|---|---|---|
| FutC | WP_080473865.1 | Helicobacter pylori |
| | | ATCC 26695 |
| wbgL | ADN43847 | E. coli 0126 |
| Smob | WP_126455392.1 | Sulfuriflexus mobilis |
| FucT2_HpUA802 | AAC99764.1 | Helicobacter pylori |
| FucT2_EcO126t | ABE98421.1 | Escherichia coli |
| FucT2_Hm12198 | CBG40460.1 | Helicobacter mustelae 12198 |
| FucT2_Pm9515 | ABM71599.1 | Brochlorococcus marinus str. |
| | | MIT 9515 |
| FucT2_HpF57 | BAJ59215.1 | Helicobacter pylori F57 |
| FucT54 | ADE13114.1 | Sideroxydans lithotrophicus |
| | | ES-11 |
| Mtun | WP_031437198.1 | Methylobacter tundripaludum |

In embodiments the cell of the present invention further comprises a recombinant nucleic acid sequence encoding the α-1,2-fucosyltransferase(s) from Sulfuriflexus mobiles with an amino acid sequence according to SEQ ID NO: 32, or a functional homologue thereof with an amino acid sequence that is at least 80% identical to SEQ ID NO: 32.

Below are non-limiting examples of genetically modified strains according to the present invention with specific combinations of glycosyl transferases that will lead to production of LNDFH-I using lactose as initial substrate.

In a non-limiting example, LgtA from Neisseria meningitidis is used in combination with galTK from Helicobacter pylori, Smob from Sulfuriflexus mobilis and Med1 from Mediterranea sp. An20 to produce LNDFH-I, starting from lactose as initial substrate.

In a non-limiting example, galTK from Helicobacter pylori is used in combination with Smob from Sulfuriflexus mobilis and Med1 from Mediterranea sp. An20 to produce LNDFH-I, starting from LNT-II as initial substrate.

### Glycosyl-Donor—Nucleotide-Activated Sugar Pathways

When carrying out the method of this invention, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid. The genetically engineered cell according to the present invention can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In one embodiment of the current invention, the genetically engineered cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically engineered cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Colanic Acid Gene Cluster

For the production of fucosylated HMOs, the de novo GDP-fucose pathway is important to ensure presence of sufficient GDP-fucose. The colanic acid gene cluster of Escherichia coli encodes most of the enzymes involved in the de novo synthesis of GDP-fucose (gmd, wcaG, wcaH, wcal, manB, manC), whereas one or several of the genes downstream of GDP-L-fucose such as wcaJ, which are responsible for the production of the extracellular polysaccharide colanic acid, a major oligosaccharide of the bacterial cell wall, can be deleted to prevent conversion of GDP-fucose to colanic acid.

To secure sufficient amounts of GDP-fucose the promoter of the native colanic acid gene cluster may be exchanged with a stronger promoter, generating a recombinant colanic acid gene cluster, to drive additional production of GDP-fucose. Furthermore, an extra copy of the colanic acid gene cluster or selected genes thereof can be introduced in the genetically engineered cells as described in the examples.

In embodiments, the colanic acid gene cluster may be expressed from its native genomic locus. The expression may be actively modulated. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

The de novo GDP-fucose pathway genes responsible for the formation of GDP-fucose comprises or consists of the following genes:
i) manA which encodes the protein mannose-6 phosphate isomerase (EC 5.3.1.8, UniProt accession nr. P00946), which facilitates the interconversion of fructose 6-phosphate (F6P) and mannose-6-phosphate;
ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate;
iii) manC which encodes the protein mannose-1-phosphate guanylyltransferase guanylyltransferase (EC:2.7.7.13, UniProt accession nr P24174), which is involved in the biosynthesis of GDP-mannose through synthesis of GDP-mannose from GTP and α-D-mannose-1-phosphate;
iv) gmd which encodes the protein GDP-mannose-4,6-dehydratase (UniProt accession nr P0AC88), which catalyzes the conversion of GDP-mannose to GDP-4-dehydro-6-deoxy-D-mannose;
v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster (e.g. as identified in SEQ ID NO: 3 or a functional variant thereof) and/or one or more genes of the de novo GDP-fucose pathway selected from the group consisting of manA, manB, manC, gmd and wcaG.

### Lactose Permease

Lactose permease is a membrane protein which is a member of the major facilitator superfamily and can be classified as a symporter, which uses the proton gradient towards the cell to transport β-galactosides such as lactose in the same direction into the cell. In oligosaccharide production, especially in the production of human milk oligosaccharides (HMOs), lactose is often the initial substrate being decorated to produce any HMO of interest in a bioconversion that happens in the cell interior. Thus, in the production of HMOs, there is a desire to be able to import lactose into the cell, e.g., by expression and/or overexpression of a lactose permease such as lacY of E. coli K.-12.

In embodiments, the lactose permease is as shown in SEQ ID NO: 6, or a functional homologue thereof having an amino acid sequence which is at least 80% identical, such as at least 85%, 90% or 95% identical to SEQ ID NO: 6.

In embodiments, the expression of the lactose permease is regulated by a promoter according to the present invention.

### β-Galactosidase

A host cell suitable for HMO production, e.g., E. coli, may comprise an endogenous β-galactosidase gene or an exogenous β-galactosidase gene, e.g., E. coli comprises an endogenous lacZ gene (e.g., GenBank Accession Number V00296 (GI:41901)). For the purposes of the invention, when producing an HMO, the genetically engineered cell is genetically manipulated to either not comprise any β-galactosidase gene or to comprise a β-galactosidase gene that is inactivated. The gene may be inactivated by a complete or partial deletion of the corresponding nucleic acid sequence from the bacterial genome, or the gene sequence is mutated in the way that it is not transcribed, or, if transcribed, the transcript is not translated or if translated to a protein (i.e., β-galactosidase), the protein does not have the corresponding enzymatic activity. In this way the HMO-producing bacterium accumulates an increased intracellular lactose pool which is beneficial for the production of HMOs.

### Transporter Proteins

The oligosaccharide product, such as the HMO produced by the cell, can be accumulated both in the intra- and the extracellular matrix. The product can be transported to the supernatant in a passive way, i.e., it diffuses outside across the cell membrane. The more complex HMO products may remain in the cell, which is likely to eventually impair cellular growth, thereby affecting the possible total yield of the product from a single fermentation. The HMO transport can be facilitated by major facilitator superfamily transporter proteins that promote the effluence of sugar derivatives from the cell to the supernatant. The major facilitator superfamily transporter can be present exogenously or endogenously and is overexpressed under the conditions of the fermentation to enhance the export of the oligosaccharide derivative (HMO) produced. The specificity towards the sugar moiety of the product to be secreted can be altered by mutation by means of known recombinant DNA techniques.

Thus, the genetically engineered cell according to the present invention can further comprise a nucleic acid sequence encoding a major facilitator superfamily transporter protein capable of exporting the fucosylated human milk oligosaccharide product or products, such a transporter protein can for example be a member of the major facilitator superfamily transporters.

In the resent years, several new and efficient major facilitator superfamily transporter proteins have been identified, each having specificity for different recombinantly produced HMOs and development of recombinant cells expressing said proteins are advantageous for high scale industrial HMO manufacturing.

Thus, in one or more exemplary embodiments, the genetically engineered cell according to the present invention further comprises a gene product that acts as an LNFP-II, LNFP-III or LNDFH-I transporter. The gene product that acts as an LNFP-II, LNFP-III or LNDFH-I transporter may be encoded by a recombinant nucleic acid sequence that is expressed in the genetically engineered cell. The recombinant nucleic acid sequence encoding the LNFP-II, LNFP-III or LNDFH-I transporter, may be integrated into the genome of the genetically engineered cell, or expressed using a plasmid.

In one embodiment, the genetically engineered cell of the invention comprises a nucleic acid sequence encoding a major facilitator superfamily transporter protein capable of exporting the fucosylated human milk oligosaccharide product into the extracellular medium, in particular, the transporters with specificity towards exporting complex fucosylated HMOs, such as LNFP-II, LNFP-III or LNDFH-I, are preferred.

### The Genetically Engineered Cell

In the present context, the terms “a genetically engineered cell” and “a genetically engineered cell” are used interchangeably. As used herein “a genetically engineered cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, and/or metabolic pathway engineering and inclusion of transporters as described in the above sections, which the skilled person will know how to combine into a genetically engineered cell capable of producing one or more fucosylated HMOs, preferably the one or more fucosylated HMOs is selected from the group consisting of LNFP-II, LNFP-III and LNDFH-I.

In embodiments of the present disclosure, the complex fucosylated human milk oligosaccharide (HMO) produced by the cell is selected from the group consisting of LNFP-II, LNFP-III and LNDFH-I. In general, LNFP-II, LNFP-III and LNDFH-I are not produced by the same cell but requires different genetic modifications in the background strain to provide either a LNT, LNnT or LNFP-I strain, respectively. Accordingly, production of LNFP-II, LNFP-III or LNDFH-I requires the presence of three or more glycosyltransferase activities, if starting from lactose as the initial substrate or two or more glycosyltransferase activities, if starting from LNT-II as the initial substrate.

In this regard, a cell of the present invention expressing a β-1,3-N-acetyl-glucosaminyl-transferase and a β-1,3-galactosyltransferase in combination with the α-1,3(4)-fucosyltransferase of the invention, is capable of producing the complex fucosylated HMO, LNFP-II.

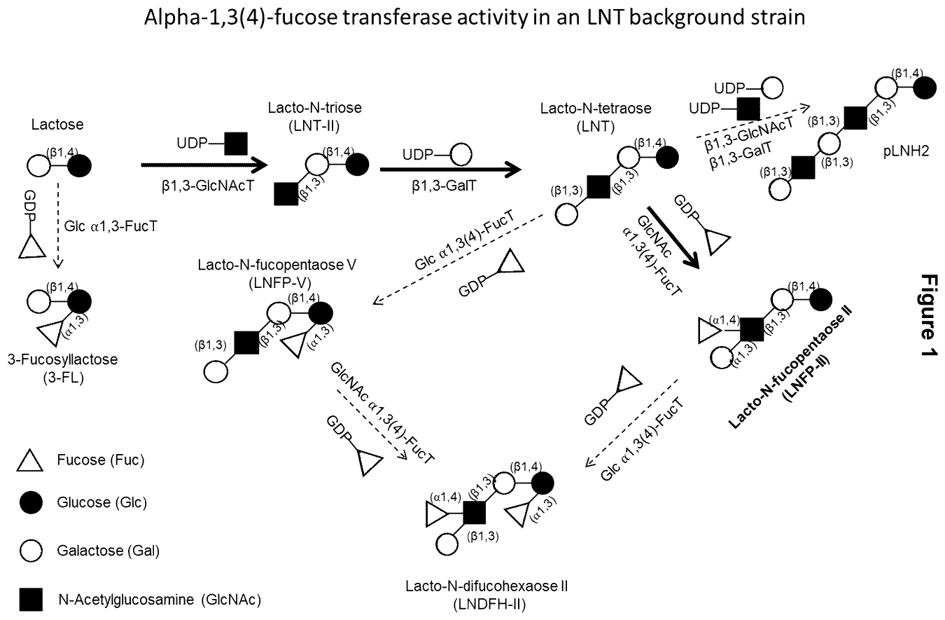

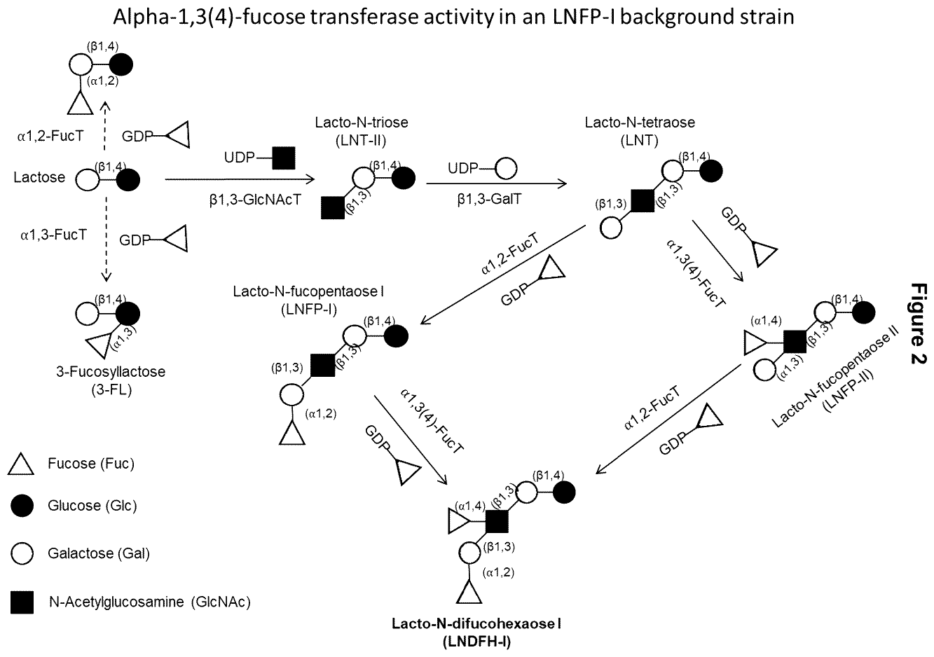

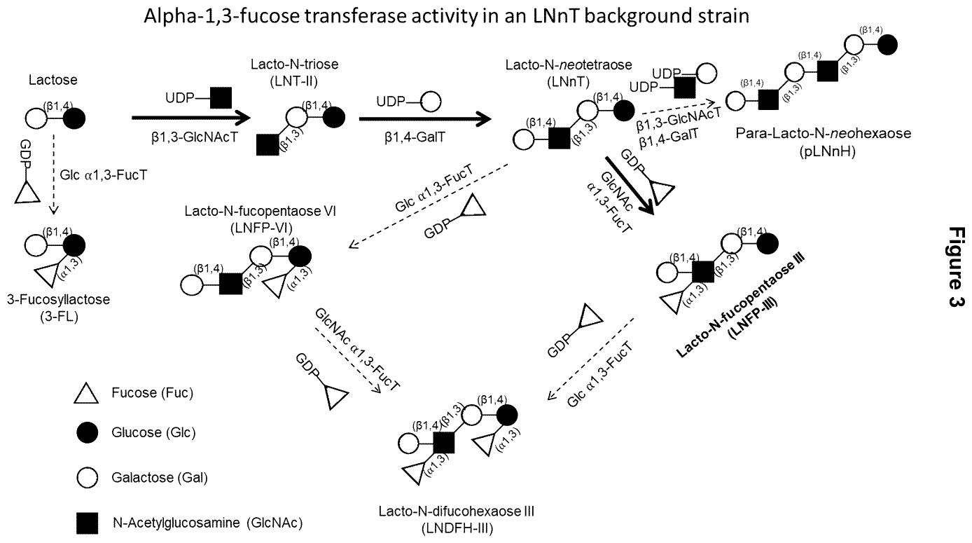

The HMOs produced by a strain containing the α-1,3(4)-fucosyltransferase described herein can be mixtures of HMOs that comprise the precursor HMOs, such as LNT, LNnT or LNFP-I, and/or by-product oligosaccharides or by-product HMOs, such as 2′FL, 3FL, DFL, pLNH2 or pLNnH. pLNH2 is a potential oligosaccharide by-product as illustrated in FIG. 1, it is however not officially reported as an HMO although it may very well be present in human mothers' milk in small amounts.

In preferred embodiments the mixtures are however essentially free of the alternative complex fucosylated HMOs LNFP-V, LNFP-VI and/or LNDFH-II

In additional embodiments, the cell of the present invention produces a mixture of HMOs comprising LNFP-II, LNT, and pLNH2 or LNFP-II, LNnT and 3FL or LNFP-II, LNT, 3FL and pLNH2, or LNFP-II, LNT, 3FL, LNT-II and pLNH2.

In additional embodiments, the cell of the present invention produces a mixture of HMOs comprising LNFP-III and LNnT, LNFP-III, LNnT and pLNnH, or LNFP-III and 3FL.

In additional embodiments, the cell of the present invention produces a mixture of HMOs comprising LNFP-I, 2′FL and LNDFH-I.

In preferred embodiments, the engineered cell is capable of producing one or more fucosylated HMOs according to any of the preceding claims, wherein the cell produces a mixture of HMOs essentially consisting of
a) LNFP-II, pLNH2 and LNT, or
b) LNFP-II, LNT and 3FL, or
c) LNFP-II, LNT, 3FL and pLNH2, or
d) LNT-II, LNFP-II, LNnT, 3FL and pLNH2, or
e) LNFP-III and LNnT, or
f) LNFP-III, LNnT and pLNnH, or
g) LNFP-III and 3FL or
h) LNFP-I, 2′FL and LNDFH-I.

In embodiments, the total HMOs produced by said cell is essentially free of LNFP-VI and/or LNDFH-II. In the present invention, “essentially free of LNFP-VI and/or LNDFH-III”, is to be understood as a content of LNFP-VI and/or LNDFH-III in the total HMO produced by the cell that is less than 1%. In the present invention, “essentially free of LNFP-V and/or LNDFH-II”, is to be understood as a content of LNFP-V and/or LNDFH-II in the total HMO produced by the cell that is less than 1%. In the present invention, “essentially free of 3FL”, is to be understood as a content of 3FL in the total HMO produced by the cell that is less than 1%.

In preferred embodiments the genetically engineered cell capable of producing one or more fucosylated HMOs, preferably the one or more fucosylated HMOs selected from the group consisting of LNFP-II, LNFP-III and LNDFH-I, comprises a recombinant nucleic acid sequence encoding a fucosyltransferase with α-1,3(4)-fucosyltransferase activity, wherein said enzyme is Med1 with an amino acid sequence according to SEQ ID NO: 1, or a functional homologue thereof with an amino acid sequence that is at least 80% identical to SEQ ID NO: 1.

### Host Cells

In embodiments, the engineered cell is a microorganism. The genetically engineered cell is preferably a microbial cell, such as a prokaryotic cell or an eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells, such as Komagataella, Kluyveromyces, Yarrowia, Pichia, Saccaromyces, Schizosaccharomyces or Hansenula or from a filamentous fungus of the genera Aspergillus, Fusarium or Thricoderma.

In one or more exemplary embodiments, the genetically engineered cell is selected from the group consisting of Escherichia sp., Bacillus sp., lactobacillus sp., Corynebacterium sp. and Campylobacter sp.

In one or more exemplary embodiments, the genetically engineered cell is selected from the group consisting of Escherichia coli, Bacillus subtilis, lactobacillus lactis, Corynebacterium glutamicum, Yarrowia lipolytica, Pichia pastoris, and Saccharomyces cerevisiae.

In one or more exemplary embodiments, the genetically engineered cell is B. subtilis.

In one or more exemplary embodiments, the genetically engineered cell is S. Cerevisiae or P pastoris.

In one or more exemplary embodiments, the genetically engineered cell is Escherichia coli.

In one or more exemplary embodiments, the invention relates to a genetically engineered cell, wherein the cell is derived from the E. coli K-12 strain or DE3.

### HMO Mixtures Produced by the Cell

The genetically engineered cell comprising more than one glycosyltransferase described herein will generally produce a mixture of HMOs as a result of the multistep process towards the final HMO product. In the production of LNFP-II from lactose as the initial substrate, it is possible that LNT-II, LNT, LNFP-II, and pLNH2, and potentially also 3FL (fucosylated lactose), LNFP-V and LNDFH-II are present at the end of the cultivation.

In the production of LNDFH-I from lactose as the initial substrate, it is possible that 2′FL (fucosylated lactose), LNT-II, LNT, LNFP-I and LNDFH-I, and potentially also, 3FL, LNFP-II, LNFP-V, LNDFH-II and pLNH2 are present at the end of the cultivation.

In the production of LNFP-III from lactose as the initial substrate, it is possible that LNT-II, LNnT, LNFP-III, and pLNnH, and potentially also 3FL (fucosylated lactose), LNFP-VI and LNDFH-III are present at the end of the cultivation.

In that regard, a mixture of HMOs may consists essentially of a) LNFP-II, pLNH2 and LNT, or b) LNFP-II, LNT and 3FL, or c) LNFP-II, LNT, 3FL and pLNH2, or d) LNT-II, LNFP-II, LNT, 3FL and pLNH2, or e) LNFP-III and LNnT, or f) LNFP-III, LNnT and pLNnH, or g) LNFP-III and 3FL or h) LNFP-I, 2′FL and LNDFH-I.

The HMO products produced by the methods disclosed herein can be described by their molar % in a mixture of HMOs. The “molar % of an HMO” as described herein is understood as the molar % of an HMOs from the total HMO produced.

In one embodiment of the invention, following cultivation of the genetically engineered cell as described herein, the mixture of HMOs consists essentially of LNFP-II and LNT in combination with one or more of 3FL and/or LNT-II and/or pLNH2.

In one embodiment of the invention, following cultivation of the genetically engineered cell as described herein, the mixture of HMOs consists essentially of 3-35% LNFP-II, 45-85% LNT, 0-20% 3FL, 0-15% LNT-II and 0-5% pLNH2, with the total molar HMO content in the mixture adding up to 100%.

In embodiments of the present invention, the fucosylated human milk oligosaccharide (HMO) produced by the cell is LNFP-II, such as exclusively or essentially exclusively LNFP-II. In a further embodiment of the present invention, at least 5%, such as at least 6%, 7%, 8% 9%, 10%, 12%, 15%, 20%, 25% or 30% of the molar content of the total HMOs produced by said cell is LNFP-II. Preferably, at least 25% of the molar content of the total HMOs produced by said cell is LNFP-II. In further embodiments, at least 75% of the molar content of the total HMOs produced by said cell is LNFP-II and LNT. In additional embodiments of the invention, less than 1%, such as 0.0%, 0.1%, 0.2%, 0.3%, 0.4%, 0.5%, 0.6%, 0.7%, 0.8%, 0.9% or 0.99% of the molar content of the total HMOs produced by the cell is LNFP-V. In additional embodiments of the invention, less than 5%, such as less than 4%, such as less than 3%, such as less than 2%, such as less than 1%, such as 0.0%, 0.5%, 0.8%, 1.0%, 1.5%, 2.0%, 2.5% of the molar content of the total HMOs produced by the cell is LNDFH-II. In additional embodiments less than 5% of the molar content of the total HMOs produced by the cell is an alternative complex fucosylated HMO. In additional embodiments less than 25%, such as less than 50%, such as less than 15%, such as less than 10%, such as less than 5%, such as less than 1% of the molar content of the total HMOs produced by the cell is a non-complex fucosylated HMO, such as 3FL. In the present context, an alternative fucosylated HMO is considered one or more fucosylated HMO which is not LNFP-II. The alternative fucosylated HMO(s) may be selected from the groups consisting of 3FL, DFL, LNFP-V and LNDFH-II. The alternative complex fucosylated HMO may be selected from the groups consisting of LNFP-V and LNDFH-II. In one embodiment of the invention, following cultivation of the genetically engineered cell as described herein, the mixture of HMOs consists essentially of LNDFH-I, LNFP-I, and 2′FL.

In one embodiment of the invention, following cultivation of the genetically engineered cell as described herein, the mixture of HMOs consists essentially of 7-15% LNDFH-1, 65-90% LNFP-I and 5-20% 2′FL with the total molar HMO content in the mixture adding up to 100%.

In a further embodiment of the present invention, at least 5% such as at least 6%, 7%, 8%, 9%, 10% or 11% of the molar content of the total HMOs produced by said cell is LNDFH-I. In further embodiments, at least 80% of the molar content of the total HMOs produced by said cell is LNFP-I and LNDFH-I. In additional embodiments of the invention, less than 1%, such as 0.0%, 0.1%, 0.2%, 0.3%, 0.4%, 0.5%, 0.6%, 0.7%, 0.8%, 0.9% or 0.99% of the molar content of the total HMOs produced by the cell is LNFP-V and/or LNDFH-II.

In additional embodiments less than 5% of the molar content of the total HMOs produced by the cell is an alternative fucosylated HMO. In the present context, when the primarily produced HMOs are LNDFH-I and LNFP-I, the alternative fucosylated HMO(s) is considered one or more fucosylated HMO which is not LNDFH-I or LNFP-I. Accordingly, when the primarily produced HMOs are LNDFH-I and LNFP-I, the alternative fucosylated HMO(s) may be selected from the group consisting of 3FL, 2′FL, DFL, LNFP-V and LNDFH-II.

In one embodiment of the invention, following cultivation of the genetically engineered cell as described herein, the mixture of HMOs consists essentially of LNFP-III and LNnT, with low amounts of 3FL, or pLNnH, such as below 10% total molar HMO content in the composition.

In one embodiment of the invention, following cultivation of the genetically engineered cell as described herein, the mixture of HMOs consists essentially of 50-99 molar % LNFP-III, 0-45 molar % LNnT, 0-20% 3FL and 0-10% pLNnH, in total adding up to 100% molar content.

In embodiments of the present invention, the fucosylated human milk oligosaccharide (HMO) produced by the cell is LNFP-III, such as exclusively or essentially exclusively LNFP-III. In a further embodiment of the present invention, at least 30%, such as at least 50%, 60%, 70%, 75%, 80%, 85%, 90%, 95%, 98% or 99% of the molar content of the total HMOs produced by said cell is LNFP-III. Preferably, at least 75% of the molar content of the total HMOs produced by said cell is LNFP-III.

In further embodiments, at least 90% of the molar content of the total HMOs produced by said cell is LNFP-III and LNnT. In additional embodiments of the invention, less than 1%, such as 0.0%, 0.1%, 0.2%, 0.3%, 0.4%, 0.5%, 0.6%, 0.7%, 0.8%, 0.9% or 0.99% of the molar content of the total HMOs produced by the cell is LNFP-VI. In additional embodiments of the invention, less than 5%, such as less than 4%, such as less than 3%, such as less than 2%, such as less than 1%, such as 0.0%, 0.5%, 0.8%, 1.0%, 1.5%, 2.0%, 2.5% of the molar content of the total HMOs produced by the cell is LNDFH-III. In additional embodiments less than 5% of the molar content of the total HMOs produced by the cell is an alternative complex fucosylated HMO. In additional embodiments less than 20%, such as less than 15%, such as less than 10%, such as less than 5%, such as less than 1% of the molar content of the total HMOs produced by the cell is a non-complex fucosylated HMO, such as FL. In the present context, an alternative fucosylated HMO is considered one or more fucosylated HMO which is not LNFP-III. The alternative fucosylated HMO(s) may be selected from the groups consisting of 3FL, DFL, LNFP-VI and LNDFH-III. The alternative complex fucosylated HMO may be selected from the groups consisting of LNFP-VI and LNDFH-III

In some embodiments, the genetically engineered cell of the present invention expresses Med1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, and the molar % content of LNFP-III produced by the genetically engineered cell is above 50%, such as above 60%, such as above 70%, such as above 75%, such as above 85% of the total HMO.

In some embodiments, the genetically engineered cell of the present invention expresses Med1 comprising or consisting of the amino acid sequence of SEQ ID NO: 1, or an amino acid sequence with at least 80%, such as at least 85%, such as at least 90%, such as at least 95%, or such as at least 99% sequence identity to SEQ ID NO: 1, and the molar % content of LNFP-II produced by the genetically engineered cell is above 5%, such as above 10%, such as above 20%, such as above 30% of the total HMO.

In some embodiments, the genetically engineered cell of the present invention expresses Med1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, and the molar % content of LNDFH-I produced by the genetically engineered cell is above 5%, such as above 6%, such as above 8% such as above 9% of the total HMO.

The molar % of individual HMO components supported are supported by experimental data from Example 1-5 which shows exemplary HMO mixture ranges for genetically engineered cell expressing the fucosyltransferase Med1.

### A Recombinant Nucleic Acid Sequence

The present invention relates to a genetically engineered cell comprising a recombinant nucleic acid sequence encoding a fucosyltransferase with α-1,3(4)-fucosyltransferase activity, specifically the α-1,3(4)-fucosyltransferase Med1, and wherein said cell produces Human Milk Oligosaccharides (HMO), preferably, the fucosylated HMOs is/are selected from fucosylated HMOs comprising at least five or six monosaccharide units, more preferably the one or more fucosylated HMOs is selected from the group consisting of LNFP-II, LNFP-III and LNDFH-I.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The invention also relates to a nucleic acid construct comprising a coding nucleic sequence, i.e. recombinant DNA sequence of a gene of interest, e.g., a fucosyltransferase gene, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the invention may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular, a DNA segment, which is intended to be inserted into a target cell, e.g., a bacterial cell, to modify expression of a gene of the genome or expression of a gene/coding DNA sequence which may be included in the construct. Thus, in embodiments, the present invention relates to a nucleic acid construct comprising a recombinant nucleic acid sequence encoding a fucosyltransferase, wherein said recombinant nucleic acid sequence is a nucleic acid sequences encoding Med1, such as a nucleic acid sequence according to SEQ ID NO: 2, or functional variants thereof.

Accordingly, in one aspect of the invention, the genetically engineered cell comprises a recombinant nucleic acid sequence encoding a fucosyltransferase with α-1,3(4)-fucosyltransferase activity, which is capable of producing at least 50%, preferably at least 80%, LNFP-III of the total molar HMO content produced by the cell.

In this regard, a cell of the present invention expressing a β-1,3-N-acetyl-glucosaminyl-transferase and β-1,4-galactosyltransferase in combination with the α-1,3(4)-fucosyltransferase of the invention, is capable of producing the complex fucosylated HMO, LNFP-III.

In one aspect of the invention, the genetically engineered cell comprises a recombinant nucleic acid sequence encoding a fucosyltransferase with α-1,3(4)-fucosyltransferase activity, which is capable of producing at least 5% LNFP-II of the total molar HMO content produced by the cell.

In one aspect of the invention, the genetically engineered cell comprises a recombinant nucleic acid sequence encoding a fucosyltransferase with α-1,3(4)-fucosyltransferase activity, which is capable of producing at least 8% LNDFH-I of the total molar HMO content produced by the cell.

Furthermore, a cell expressing a β-1,3-N-acetyl-glucosaminyl-transferase, a β-1,3-galactosyltransferase in combination with an α-1,2-fucosyltransferase and the α-1,3(4)-fucosyltransferase of the invention, is capable of producing complex fucosylated HMO, LNDFH-I in combination with the complex fucosylated HMO, LNFP-I.

The genetically engineered cell according to the present invention may also comprise multiple copies of the recombinant nucleic acid sequence encoding a fucosyltransferase. Enhancing the copy number of the fucosyltransferase Med1 was shown in Example 1 and 2 to change the ratio of the produced HMOs and enhance the level of LNFP-II and LNFP-III produced, respectively. In specific, Example 2 discloses that increasing the copy number of Med1 to two genetic copies (stain Med1_2) increased the LNFP-III production, while further increase in expression, through additional expression of Med1 from high copy plasmid resulted in a slight decrease in LNFP-II level while a sudden increase in production of 3FL was observed. In addition, it was observed for the high expressing Med1 strain that all the LNnT produced by the cell was fucosylated.

Furthermore, it is disclosed in example 1 that increasing the copy number of Med1 and the fucosyltransferase Para1 to two genetic copies increased the LNFP-II production by 10% from 5% to 15% for Med1, whereas Para1 only increased LNFP-II formation by 1%, whereas the amount of the difucosylated LNDFH-II for Para1 was increased from 2 to 11%, clearly indicating that Para1 has equal preference for fucosylating the GlcNAc and the Glc moiety of LNT, whereas Med1 only possess activity towards the GlcNAc moiety. Further increase in Med1 expression increases the LNFP-II level from 15% to 30%.

Furthermore, the increase in LNFP-II or LNFP-III observed when enhancing the expression of Med1, also resulted in a shift in the amount of each of HMOs produced by the cell, as is evident from examples 1 to 3.

Accordingly, the copy number variation may be used in the production to tailor specific HMOs mixtures, in this case a mixture comprising LNFP-III, LNnT and/or 3FL, a mixture comprising LNFP-II, LNT and 3FL or pLNH2, or a mixture comprising LNDFH-I, LNFP-I and 2′FL in different ratios, depending on the need for the specific product.

Accordingly, in embodiments, the genetically engineered cell of the present invention comprises one, two, three or more genomic copies of the recombinant nucleic acid sequence encoding the glycosyltransferase Med1 with an amino acid sequence according to SEQ ID NO: 1, or a functional homologue thereof with an amino acid sequence that is at least 80% identical to SEQ ID NO: 1.

In further embodiments, the recombinant nucleic acid sequence encoding the glycosyltransferase Med1 with an amino acid sequence according to SEQ ID NO: 1 or a functional homologue thereof with an amino acid sequence that is at least 80% identical to SEQ ID NO: 1, is encoded on a plasmid. In additional embodiments, the plasmid is a high copy number plasmid, preferably, a pUC57 plasmid.

In further embodiments, the genetically engineered cell according to the present invention comprises one, two, three or more genomic copies and/or a plasmid borne copy of the recombinant nucleic acid sequence encoding the glycosyltransferase Med1 with an amino acid sequence according to SEQ ID NO: 1, or a functional homologue thereof with an amino acid sequence that is at least 80% identical to SEQ ID NO: 1.

One embodiment of the invention relates to a nucleic acid construct comprising a recombinant nucleic acid sequence encoding a fucosyltransferase, wherein said recombinant nucleic acid sequence is Med1 comprising or consisting of the nucleic acid sequence of SEQ ID NO: 2 or a nucleic acid sequence with at least 80%, such as at least 85%, such as at least 90%, such as at least 95%, or such as at least 99% sequence identity to SEQ ID NO: 2.

Preferably, the fucosyltransferase encoding sequence is under the control of a promoter sequence selected from promotor sequences with a nucleic acid sequence as identified in Table 4.

TABLE 4
Selected promoter sequences

| | % Activity | | | Seq ID |
|---|---|---|---|---|
| Promoter name | relative to PglpF* | Strength | Reference | in appl. |
| PmglB_70UTR_SD8 | 291% | high | WO2020255054 | 7 |
| PmglB_70UTR_SD10 | 233-281% | high | WO2020255054 | 8 |
| PmglB_54UTR | 197% | high | WO2020255054 | 9 |
| Plac_70UTR | 182-220% | high | WO2019123324 | 10 |
| PmglB_70UTR_SD9 | 180-226% | high | WO2020255054 | 11 |
| PmglB_70UTR_SD4 | 153%-353% | high | WO2020255054 | 12 |
| PmglB_70UTR_SD5 | 146-152% | high | WO2020255054 | 13 |
| PglpF_SD4 | 140-161% | high | WO2019123324 | 14 |
| PmglB_70UTR_SD7 | 127-173% | high | WO2019123324 | 15 |
| PmglB_70UTR | 124-234% | high | WO2020255054 | 16 |
| PglpA_70UTR | 102-179% | high | WO2019123324 | 17 |
| PglpT_70UTR | 102-240% | high | WO2019123324 | 18 |
| PglpF | 100% | high | WO2019123324 | 19 |
| PglpF_SD10 | 88-96% | high | WO2019123324 | 20 |
| PglpF_SD5 | 82-91% | high | WO2019123324 | 21 |
| PglpF_SD8 | 81-82% | high | WO2019123324 | 22 |
| PmglB_16UTR | 78-171% | high | WO2019123324 | 23 |
| PglpF_SD9 | 73-93% | middle | WO2019123324 | 24 |
| PglpF_SD7 | 47-57% | middle | WO2019123324 | 25 |
| PglpF_SD6 | 46-47% | middle | WO2019123324 | 26 |
| PglpA_16UTR | 38-64% | middle | WO2019123324 | 27 |
| Plac_wt* | 15-28% | low | WO2019123324 | 28 |
| PglpF_SD3 | 9% | low | WO2019123324 | 29 |
| PglpF_SD1 | 5% | low | WO2019123324 | 30 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically engineered cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assessed using a lacZ enzyme assay where R-galactosidase activity is assayed as described previously (see e.g., Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU. In preferred embodiments, the expression of said nucleic acid sequences are under control of a strong promoter selected from the group consisting of SEQ ID NOs 7, 8, 9, 10, 11, 12, 13, 14, 15, 16, 17 and 18.

In embodiments the expression of said nucleic acid sequences of the present invention is under control of a PglpF (SEQ ID NO: 19) or Plac (SEQ ID NO: 28) promoter or PmgIB_UTR70 (SEQ ID NO: 16) or PglpA_70UTR (SEQ ID NO: 17) or PglpT_70UTR (SEQ ID NO: 18) or variants thereof such as promoters identified in Table 4, in particular, the PglpF_SD4 variant of SEQ ID NO: 14 or Plac_70UTR variant of SEQ ID NO: 10, or PmgIB_70UTR variants of SEQ ID NO: 7, 8, 9, 11, 12, 13 and 14. Further suitable variants of PglpF, PglpA_70UTR, PgIpT_70UTR and PmgIB_70UTR promoter sequences are described in or WO2019/123324 and WO2020/255054 respectively (hereby incorporated by reference).

In preferred embodiments, the recombinant nucleic acid sequences individually are under the control of one or more promoters selected from the group consisting of PglpF, Plac, PmgIB_70UTR, PglpA_70UTR and PglpT_70UTR (SEQ ID NOs: 19, 28, 16, 17 and 18, respectively) and variants thereof.

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage A or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56.; Vetcher et al., Appl Environ Microbiol. (2005); 71(4):1829-35); or positive clones, i.e., clones that carry the expression cassette, can be selected e.g., by means of a marker gene, or loss or gain of gene function.

In one or more exemplary embodiments, the present disclosure relates a recombinant nucleic acid sequence as illustrated in SEQ ID NO: 2.

In particular, the present disclosure relates to a recombinant nucleic acid sequence and/or to a functional homologue thereof having a sequence which is at least 70% identical to SEQ ID NO: 2, such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the −nobrief option) is used as the percent identity. Generally sequence identity may be calculated as follows: (Identical Residues×100)/(Length of Aligned region).

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the −nobrief option) is used as the percent identity. Generally sequence identity may be calculated as follows: (Identical Deoxyribonucleotides×100)/(Length of Aligned region).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of the amino acid sequence of Med1 with the amino acid sequence of SEQ ID NO: 1, or a recombinant nucleic acid encoding to SEQ ID NO: 2, should ideally be able to participate in the production of fucosylated HMOs, in terms of increased HMO yield, improved purity/by-product formation, reduction in biomass formation, viability of the genetically engineered cell, robustness of the genetically engineered cell according to the disclosure, or reduction in consumables needed for the production.

### Use of a Genetically Engineered Cell or Enzyme

The disclosure also relates to any commercial use of the enzyme(s), genetically engineered cell(s) or the nucleic acid construct(s) disclosed herein, such as, but not limited to, in a method for producing one or more fucosylated human milk oligosaccharide (HMO).

Accordingly, the present invention also relates to the use of a the fucosyltransferase Med1 with α-1,3(4)-fucosyltransferase activity with an amino acid sequence according to SEQ ID NO: 1 or a functional homologue thereof which amino acid sequence is at least 80% identical to SEQ ID NO: 1 activity in production of a fucosylated product. In embodiments, the fucosyltransferase of the present invention are also used in the manufacturing of a fucosylated product, wherein the fucosylated product is one or more fucosylated oligosaccharides or one or more fucosylated polypeptides. Preferably the one or more fucosylated oligosaccharides is one or more HMOs, preferably, the fucosylated HMOs is/are selected from fucosylated HMOs comprising at least five monosaccharide units preferably selected from fucosylated HMOs with an LNT and/or LNnT backbone structure, more preferably the fucosyltransferase only fucosylates the N-acetylglucoseamine (GlcNAc) moiety of LNT or LNnT.

In an exemplified embodiment, the α-1,3(4)-fucosyltransferase, the genetically engineered cell and/or the nucleic acid construct according to the invention is used in the manufacturing of one or more fucosylated HMO(s), preferably, LNFP-II, LNFP-III and LNDFH-I.

In an exemplified embodiment, the α-1,3(4)-fucosyltransferase, the genetically engineered cell and/or the nucleic acid construct according to the invention is used in the manufacturing of HMOs. Preferably, the genetically engineered cell and/or the nucleic acid construct is used in the manufacturing of the HMOs LNFP-II, LNFP-III or LNDFH-I. In addition, the genetically engineered cell and/or the nucleic acid construct is preferably used in manufacturing of a mixture of HMOs wherein the at least 5% of the molar content of the total HMOs produced by said cell is LNFP-II, wherein at least 50% of the molar content of the total HMOs produced by said cell is LNFP-II or wherein at least 8% of the molar content of the total HMOs produced by said cell is LNDFH-I.

In embodiments the fucosyltransferase, the genetically engineered cell and/or the nucleic acid construct is used in the manufacturing of one or more fucosylated HMOs, wherein the cell produces a mixture of HMOs comprising
a) LNFP-II, pLNH2 and LNT, or
b) LNFP-II, LNT and 3FL, or
c) LNFP-II, LNT, 3FL and pLNH2, or
d) LNT-II, LNFP-II, LNT, 3FL and pLNH2, or
e) LNFP-III and LNnT, or
f) LNFP-III, LNnT and pLNnH, or
g) LNFP-III and 3FL, or
h) LNDFH-I, LNFP-I, 2′FL.

Production of these HMO's may require the presence of two or more glycosyltransferase activities when starting the production from lactose or LNT-II.

### a Method for Producing Fucosylated Human Milk Oligosaccharides (HMOs)

The present invention also relates to a method for producing one or more fucosylated human milk oligosaccharides (HMOs), said method comprises culturing a genetically engineered cell according to the present invention.

One aspect is a method for producing one or more fucosylated human milk oligosaccharides (HMOs), said method comprises comprising the steps of:
a) providing a genetically modified cell as described herein; and
b) culturing the cell according to (a) in a suitable cell culture medium to produce said one or more fucosylated HMOs, and
c) optionally, purifying said one or more fucosylated HMOs.

The present invention in particular, relates to a method for producing human milk oligosaccharides (HMOs), wherein the molar % content of LNFP-III produced by the genetically engineered cell is above 50% of the total amount of HMO produced, such as above 75% of the total amount of HMO produced, or such as above 85% of the total amount of HMO produced. In embodiments, the fucosylated HMO is LNFP-III.

The present invention in particular, relates to a method for producing one or more fucosylated human milk oligosaccharide (HMO), said method comprising culturing a genetically engineered cell, said cell comprising a recombinant nucleic acid sequence encoding a fucosyltransferase with α-1,3(4)-fucosyltransferase activity, wherein said enzyme is Med1, with an amino acid sequence according to SEQ ID NO: 1 or a functional homologue thereof with an amino acid sequence that is at least 80% identical to SEQ ID NO: 1, and wherein said cell produces fucosylated HMOs that is/are selected from fucosylated HMOs comprising at least five monosaccharide units.

In a further embodiment of the method of the present invention, the produced fucosylated HMOs is/are selected from fucosylated HMOs with an LNT and/or LNnT backbone structure. In additional embodiments of the method of the present invention, the fucosyltransferase only fucosylates the N-acetylglucoseamine (GlcNAc) moiety of LNT or LNnT. In a preferred embodiment of the method of the present invention, the one or more fucosylated HMOs is selected from the group consisting of LNFP-II, LNFP-III and LNDFH-I. In embodiments at least one of the HMOs produced with the method of the present invention is LNFP-II, LNFP-III or LNDFH-I.

A further embodiment of the invention relates to a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,3-galactosyltransferase or β-1,4-galactosyltransferase activity; and
c. a recombinant nucleic acid sequence encoding the fucosyltransferase Med1 with an amino acid sequence according to SEQ ID NO: 1 or a functional homologue thereof with an amino acid sequence that is at least 80% identical to SEQ ID NO: 1.

Further genetic modifications can e.g., be selected from inclusion of additional glycosyltransferases and/or metabolic pathway engineering, and inclusion of MFS transporters, as described in the above sections, which the skilled person will know how to combine into a genetically engineered cell capable of producing one or more fucosylated HMO's.

The method particularly comprises culturing a genetically engineered cell that produces a fucosylated HMO, wherein the LNFP-III content produced by said cell is at least 30% of the total amount of HMO produced by the cell. The method particularly comprises culturing a genetically engineered cell that produces a fucosylated HMO, wherein the LNFP-II content produced by said cell is at least 5%, preferably at least 25% of the total amount of HMO produced by the cell. The method particularly comprises culturing a genetically engineered cell that produces a fucosylated HMO, wherein the LNDFH-I content produced by said cell is at least 5%, preferably at least 8% of the total amount of HMO produced by the cell.

The method comprising culturing a genetically engineered cell that produces a fucosylated HMO and further comprises culturing said genetically engineered cell in in the presence of an energy source (carbon source) selected from the group consisting of glucose, sucrose, fructose, xylose and glycerol.

In embodiments the method of the present invention produces a mixture of HMOs wherein the cell produces a mixture of HMOs comprising
a) LNFP-II, pLNH2 and LNT, or
b) LNFP-II, LNT and 3FL, or
c) LNFP-II, LNnT, 3FL and pLNH2, or
d) LNT-II, LNFP-II, LNnT, 3FL and pLNH2, or
e) LNFP-III and LNnT, or
f) LNFP-III, LNnT and pLNnH, or
g) LNFP-III and 3FL or
h) LNFP-I, 2′FL and LNDFH-I.

In one aspect, the method according to the present invention produces a mixture of HMO(s), wherein at least 50%, such as at least 55%, 60%, 65%, 70%, 75%, 80%, 85%, 90%, 95% or 98% of the molar content of the total amount of HMOs produced is LNFP-III.

In one aspect, the method according to the present invention produces a mixture of HMO(s), wherein at least 5%, such as at least 10%, 15%, 20%, 25% or 30% of the molar content of the total amount of HMOs produced is LNFP-II.

In one aspect, the method according to the present invention produces a mixture of HMO(s), wherein at least 5%, such as at least 6%, 7%, 8%, 9%, 10%, 11% or 12% of the molar content of the total amount of HMOs produced is LNDFH-I.

In one aspect, the method according to the present invention produces a mixture of HMO(s) comprising LNFP-III, wherein the produced mixture of HMOs is essentially free of LNFP-VI and/or LNDFH-III.

In one aspect, the method according to the present invention produces a mixture of HMO(s) comprising LNFP-II wherein the produced mixture of HMOs is essentially free of LNFP-V and/or LNDFH-II.

In one aspect, the method according to the present invention produces a mixture of HMO(s), comprising LNDFH-I, wherein the produced mixture of HMOs is essentially free of 3FL.

In one aspect, the method according to the present invention produces LNFP-III. In another aspect, the method according to the present invention produces LNFP-II. In another aspect, the method of the present invention produces LNDFH-I.

To enable the production of fucosylated HMOs in the method according to the present invention, the genetically engineered cell comprises a biosynthetic pathway for making a fucose sugar nucleotide e.g., GDP-fucose.

In preferred embodiments of the methods of the present invention, the genetically engineered cell comprises an upregulated biosynthetic pathway for making a fucose sugar nucleotide. Preferably, in methods of the present invention, the fucose sugar nucleotide is GDP-Fucose. Thus, in methods of the present invention the sugar nucleotide pathway is expressed and/or upregulated in the genetically engineered cell, wherein the GDP-fucose pathway is encoded by the colanic acid gene cluster (CA) from E. coli of SEQ ID NO: 3. In methods of the present invention, the upregulation of the GDP-fucose pathway is obtained by integration of one or more copies of the colanic acid gene cluster (CA) from E. coli of SEQ ID NO: 3 into the genome of the host cell.

The method of the present invention comprises providing a glycosyl donor, which is synthesized separately by one or more genetically engineered cells and/or is exogenously added to the culture medium from an alternative source.

In one aspect, the method of the present invention further comprises providing an acceptor saccharide as substrate for the HMO formation, the acceptor saccharide comprising at least two monosaccharide units, which is exogenously added to the culture medium and/or has been produced by a separate microbial fermentation.

In one aspect, the method of the present invention comprises providing an acceptor saccharide comprising at least two monosaccharide units, which is exogenously added to the culture medium and/or has been produced by a separate microbial fermentation and which is selected form lactose, LNT-II, LNT and LNnT. In a preferred embodiment the substrate for HMO formation is lactose which is fed to the culture during the fermentation of the genetically engineered cell.

The fucosylated human milk oligosaccharide (HMO) is retrieved from the culture, either from the culture medium and/or the genetically engineered cell.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding a fucosyltransferase with α-1,3(4)-fucosyltransferase activity, wherein said enzyme is Med1, with an amino acid sequence according to SEQ ID NO: 1 or a functional homologue thereof with an amino acid sequence that is at least 80% identical to SEQ ID NO: 1, and
   wherein the fucosylated HMOs is/are selected from fucosylated HMOs comprising at least five monosaccharide units, preferably the one or more fucosylated HMOs is LNFP-III.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,3-galactosyltransferase activity; and
c. a recombinant nucleic acid sequence encoding a fucosyltransferase with α-1,3(4)-fucosyltransferase activity, wherein said enzyme is Med1, with an amino acid sequence according to SEQ ID NO: 1 or a functional homologue thereof with an amino acid sequence that is at least 80% identical to SEQ ID NO: 1, and
   wherein the fucosylated HMOs is/are selected from fucosylated HMOs comprising at least five monosaccharide units, preferably the one or more fucosylated HMOs is LNFP-II.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,3-galactosyltransferase activity; and
c. a recombinant nucleic acid sequence encoding a fucosyltransferase with α-1,3(4)-fucosyltransferase activity, wherein said enzyme is Med1, with an amino acid sequence according to SEQ ID NO: 1 or a functional homologue thereof with an amino acid sequence that is at least 80% identical to SEQ ID NO: 1, and
   wherein the fucosylated HMOs is/are selected from fucosylated HMOs comprising at least five monosaccharide units, preferably the one or more fucosylated HMOs is LNDFH-I.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,3-galactosyltransferase activity; and
c. a recombinant nucleic acid sequence encoding a fucosyltransferase with α-1,2-fucosyltransferase activity; and
d. a recombinant nucleic acid sequence encoding a fucosyltransferase with α-1,3(4)-fucosyltransferase activity, wherein said enzyme is Med1, with an amino acid sequence according to SEQ ID NO: 1 or a functional homologue thereof with an amino acid sequence that is at least 80% identical to SEQ ID NO: 1, and
   wherein the fucosylated HMOs is/are selected from fucosylated HMOs comprising at least five monosaccharide units, preferably the one or more fucosylated HMOs is LNDFH-I.

Culturing or fermenting (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

The terms “manufacturing” or “manufacturing scale” or “large-scale production” or “large-scale fermentation”, are used interchangeably and in the meaning of the invention defines a fermentation with a minimum volume of 100 L, such as 1000 L, such as 10.000 L, such as 100.000 L, such as 200.000 L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behaviour of an expression system in a lab scale method, such as shake flasks, benchtop bioreactors or the deep well format described in the examples of the disclosure, does allow to predict the behaviour of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon-source can be selected from the group consisting of glucose, sucrose, fructose, xylose and glycerol. In one or more exemplary embodiments, the culturing media is supplemented with one or more energy and carbon sources selected form the group containing glycerol, sucrose and glucose. In additional embodiments, lactose is added during the cultivation of the genetically engineered cells as a substrate for the HMO formation.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

After carrying out the method of this invention, the fucosylated HMO produced can be collected from the cell culture or fermentation broth in a conventional manner.

The present invention also relates to a method for producing one or more fucosylated products, said method comprising,
a) providing a purified fucosyltransferase, Med1, with an amino acid sequence according to SEQ ID NO: 1 or a functional homologue thereof with an amino acid sequence that is at least 80% identical to SEQ ID NO: 1,
b) reacting the purified fucosyltransferase of a) with
   a. a substrate, such as an oligosaccharide or a glycosylated polypeptide, and
   b. a fucose donor, such as GDP-fucose, and
c) retrieving one or more fucosylated products from the reaction in b).

### Retrieving/Harvesting

The fucosylated human milk oligosaccharide (HMO) is retrieved from the culture medium and/or the genetically engineered cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification.

The HMOs can be purified according to the procedures known in the art, e.g., such as described in WO2017/182965. The purified HMOs can be used as nutraceuticals, pharmaceuticals, or for any other purpose, e.g., for research.

At the end of culturing, the oligosaccharide as product can be accumulated both in the intra- and the extracellular matrix.

The method according to the present invention comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose and glycerol. In one or more exemplary embodiments, the culturing media is supplemented with one or more energy and carbon sources selected form the group containing glycerol, sucrose and glucose.

### Manufactured Product

The term “manufactured product” according to the use of the genetically engineered cell or the nucleic acid construct refer to the one or more HMOs intended as the one or more product HMO(s). The various products are described above.

From the data disclosed in example 1-5, it can be seen that the Med1 fucosyltransferase was capable of producing LNFP-II, LNFP-III and LNDFH-I with minor or no side-product formation (e.g., LNFP-V, LNFP-VI, LNDFH-II or LNDFH-III) emphasising the ability and suitability of Med1 to produce specific fucosylated HMOs in large scale manufacturing.

Advantageously, the methods disclosed herein provide both a decreased ratio of by-product to product and an increased overall yield of the product (and/or HMOs in total). This, less by-product formation in relation to product formation, facilitates an elevated product production and increases efficiency of both the production and product recovery process, providing superior manufacturing procedure of HMOs.

The manufactured product may be a powder, a composition, a suspension, or a gel comprising one or more HMOs.

### Mixtures of HMOs and Compositions Thereof

Complex fucosylated HMOs, such as LNFP-II, LNFP-III and LNDFH-I and mixtures comprising LNFP-II, LNFP-III and/or LNDFH-I are highly relevant as either a nutritional supplement or as a therapeutic.

An aspect of the present disclosure is a manufactured product or composition comprising a mixture of HMOs consisting essentially of,
a) LNFP-II, pLNH2 and LNT, or
b) LNFP-II, LNT and 3FL, or
c) LNFP-II, LNT, 3FL and pLNH2, or
d) LNT-II, LNFP-II, LNT, 3FL and pLNH2, or
e) LNFP-III and LNnT, or
f) LNFP-III, LNnT and pLNnH, or
g) LNFP-III and 3FL or
h) LNDFH-I, LNFP-I and 2′FL.

As shown in example 1 and 2, copy number variation may be used in the production to tailor specific HMOs mixtures, such as those indicated above, depending on the need for the specific product.

Accordingly in embodiments, the mixture of HMOs according to the present invention consists essentially of 5-35% LNFP-II, 50-85% LNT, 0-15% LNT-II, 0-20% 3FL, and 0-5% pLNH2, with the total molar HMO content in the mixture adding up to 100%.

In further embodiments, the mixture of HMOs according to the present invention consists essentially of 50-90% LNFP-III, 0-40% LNnT, 0-20% 3FL, 0-8% pLNnH with the total molar HMO content in the mixture adding up to 100%.

In additional embodiments, the mixture of HMOs according to the present invention consists essentially of 65-75% LNFP-I and 7-15% LNDFH-I and 5-20% 2′FL with the total molar HMO content in the mixture adding up to 100%.

Mixtures of HMOs may also form part of a composition comprising additional parts, such as active pharmaceutical ingredients, food supplements, excipients, surfactants etc.

Accordingly, the invention also relates to compositions comprising HMOs with different contents of the specific HMOs.

In that regard, the composition of invention in one embodiment relates to a composition comprising a mixture of HMOs, wherein the composition comprises 5-35% LNFP-II, 50-85% LNT, 0-15% LNT-II, 0-20% 3FL, and 0-5% pLNH2, of the total molar HMO content in the composition, in total adding up to 100% molar content. A preferred embodiment is a mixture consisting essentially of 15-20% LNFP-II and 80-85% LNnT of the total molar HMO content in the composition, in total adding up to 100% molar content. Another preferred embodiment is a mixture consisting essentially of 25-35% LNFP-II, 50-60% LNnT and 15-25% 3FL of the total molar HMO content in the composition, in total adding up to 100% molar content.

In another embodiment the composition of present invention is a composition comprising a mixture of HMOs, wherein the composition comprises 50-90% LNFP-III, 0-40% LNnT, 0-20% 3FL, 0-8% pLNnH, of the total molar HMO content in the composition, in total adding up to 100% molar content.

In another embodiment the composition of present invention is a composition comprising a mixture of HMOs, wherein the composition comprises 65-75% LNFP-I and 7-15% LNDFH-I and 5-20% 2′FL of the total molar HMO content in the composition, in total adding up to 100% molar content. A preferred embodiment is a mixture consisting essentially of such as 75% LNFP-I, 10% LNDFH-I and 15% 2′FL of the total molar HMO content in the composition, in total adding up to 100% molar content.

Accordingly, the present invention also relates to the use of a composition comprising a mixture of HMOs, wherein the composition comprises LNFP-II, pLNH2 and LNT, LNFP-II, LNT and 3FL, LNFP-II, LNnT, 3FL and pLNH2, or LNT-II, LNFP-II, LNnT, 3FL and pLNH2, and wherein further the composition is essentially free of LNFP-V and has less than 5% LNDFH-II in an infant formula, a dietary supplement or medical nutrition.

In addition, the present invention also relates to the use of a composition comprising a mixture of HMOs, wherein the composition comprises or consists essentially of a) LNFP-III and LNnT, LNFP-III and 3FL, b) LNFP-III, LNnT and pLNnH or c) LNFP-III, LNnT, and 3FL and wherein further the composition is essentially free of LNFP-VI and has less than 5% LNDFH-III in an infant formula, a dietary supplement or medical nutrition.

In addition, the present invention also relates to the use of a composition comprising a mixture of HMOs, wherein the composition comprises or consists essentially of LNFP-I, 2′FL and LNDFH-I and wherein further the composition is essentially free of LNFP-V and has less than 5% LNDFH-II in an infant formula, a dietary supplement or medical nutrition.

In embodiments, the composition comprising a mixture of HMOs is a pharmaceutical composition.

### Use of HMO Mixtures and Compositions

Clinical data in infants, indicate that Human Milk Oligosaccharide supplements may help to develop the desired microbiota by serving as a food source for the good bacteria in the intestine. Naturally occurring in breast milk, HMOs have evolved over thousands of years, with HMO research (clinical and preclinical) now suggesting that specific HMO's at the correct level of supplementation can provide us with unique health benefits. In particular, Human Milk Oligosaccharide supplements may help support immunity and gut health including a support of a balanced microbiome, with a potential role in cognitive development, which may open future innovation opportunities.

Accordingly, in embodiments, the invention relates to the use of a mixture or composition according to the present invention in infant nutrition.

The present invention also relates to the use of a mixture or composition according to the present invention as a dietary supplement or medical nutrition.

The mixtures or composition of HMOs produced according to the method described herein may be used to enhance the beneficial bacteria in the gut microbiome. Beneficial bacteria are for example bacteria of the Bifidobacterium sp., lactobacillus sp. or Barnesiella sp. The enhancement of beneficial bacteria may in turn lead to increased production of short chain fatty acids (SCFAs) such as acetate, propionate and butyrate, which have been shown to have many benefits in infants and young children The benefits are e.g., inhibition of pathogen bacteria, prevention of infection and diarrhea, reduced risk of allergy and metabolic disorders (see for example WO2006/130205, WO 2017/129644, WO2017/129649).

The mixtures or composition of HMOs produced according to the method described herein may be used to reduce the abundance of undesirable viruses and bacteria in the gut microbiome. Examples of pathogenic bacteria and viruses that may be reduced by the HMO mixtures described herein are including Candida albicans, Clostridium difficile, Enterococcus faecium, Escherichia coli, Helicobacter pylori, Streptococcus agalactiae, Shigella dysenteriae, Staphylococcus aureus, nora virus and rota virus. Each mixture or composition described herein can also be used to treat and/or reduce the risk of a broad range of bacterial infections of a human.

The mixtures or composition of HMOs produced according to the method described herein, may be used to increase the regeneration and viability of lyophilized probiotics, including probiotics of Bifidobacterium sp and/or lactobacillus sp., in particular increased regeneration and viability and shelf-life in an acidic environment, such as the stomach or acidic food products, is an advantage using the HMO mixtures described herein. Examples of Bifidobacterium sp which may have increased regeneration and viability are Bifidobacterium animals lactis BB12 DSM 32269, Bifidobacterium animals lactis BIF6, Bifidobacterium longum DSM 32946, Bifidobacterium longum BB536, Bifidobacterium bifidum DSMZ 32403, Bifidobacterium infantis, Bifidobacterium breve DSM 33789, Bifidobacterium infantis SP37 DSM 32687, Bifidobacterium adolescentis DSM 34065 and/or Bifidobacterium animalis ssp. animalis DSM 16284. Examples of lactobacillus sp which may have increased regeneration and viability are Lactobacillus rhamnosus GG DSM 32550, Lactobacillus rhamnosus 19070-2 DSM 26357, Lactobacillus rhamnosus GG, Lactobacillus rhamnosus LBrGG, Lactobacillus reuteri DSM 12246, Lactobacillus plantarum TIFN101, Lactobacillus gasseri Lg-36 200B FloraFit Danisco, Lactobacillus casei DSM 32382, Lactobacillus paracasei, Lactobacillus plantarum PS 128, Lactobacillus plantarum (Sacco) DSM 32383, Lactococcus lactis PAREVE, Lactobacillus paracasei ssp. Paracasei and/or Lactobacillus Probio-Tec® LGG®, Limosilactobacillus reuteri S12 DSM 33752.

In the context of the present application “Regeneration” means the process of regaining/restoring a dried bacteria's viability (i.e., “reviving” the bacterial cells by rehydration, wherein “rehydration” means restoring fluid). This process is also sometimes referred to as “reconstitution”.

In the context of the present application “Viability” is the ability of a bacterial cell to live and function as a living cell. One way of determining the viability of bacterial cells is by spreading them on an agar plate with suitable growth medium and counting the number of colonies formed after incubation for a predefined time (plate counting). Alternatively, FACS analysis may be used.

In the context of the present application “Improving the regeneration” of Bifidobacterium sp and/or Lactobacillus sp bacteria means to increase the amount (number) of Bifidobacterium sp and/or Lactobacillus sp. bacteria successfully regenerating/reviving compared to the respective control (i.e., the amount/number of Bifidobacterium sp and/or Lactobacillus sp. bacteria without the addition of HMO).

In the context of the present application “Improving the viability” of Bifidobacterium sp and/or Lactobacillus sp bacteria means to increase the amount (number) of viable Bifidobacterium sp and/or Lactobacillus sp. bacteria compared to the respective control (i.e., the amount/number of Bifidobacterium sp and/or Lactobacillus sp. bacteria without the addition of HMO).

In the context of the present application “acidic” means having a pH below 7.0 (for example, having a pH≤6.0, or ≤5.0, or ≤4.0, or ≤3.0, or in the range of 1.0-6.0, such as from 2.0 to 5.0). The pH measured in the stomach is in the range of about 1.5-3.5. The pH measured in a healthy vagina is in the range of about 3.8-5.0. The pH of fruit juices is in the range of about 2.0-4.5.

An embodiment of the present invention is a composition comprising a mixture of HMOs as described herein, in particular in the section “Mixtures of HMOs”, and one or more probiotics.

Preferably, the probiotic is a Bifidobacterium sp and/or lactobacillus sp such as any of the specific species mentioned above.

The mixtures or composition of HMOs produced according to the method described herein, may be used to extend the shelf life of probiotics, such as Bifidobacterium sp, and/or lactobacillus sp.

The mixtures or composition of HMOs produced according to the method described herein, may be used to improve the flowability of a powder or decrease the viscosity of a liquid.

The mixtures or composition of HMOs produced according to the method described herein, are used in a nutritional composition. Nutritional compositions are for example, an infant formula, a rehydration solution, or a dietary maintenance, medical nutrition or supplement for elderly individuals or immunocompromised individuals. Macronutrients such as edible fats, carbohydrates and proteins can also be included in such anti-infective compositions. Edible fats include, for example, coconut oil, soy oil and monoglycerides and diglycerides. Carbohydrates include, for example, glucose, edible lactose and hydrolysed cornstarch. Proteins include, for example, soy protein, whey, and skim milk. Vitamins and minerals (e. g. calcium, phosphorus, potassium, sodium, chloride, magnesium, manganese, iron, copper, zinc, selenium, iodine, and Vitamins A, E, D, C, and B complex) can also be included in such anti-infective compositions.

### Sequences

The current application contains a sequence listing in text format and electronical format which is hereby incorporated by reference.

An overview of the SEQ ID NOs used in the present application can be found table 4 (promoter sequences), additional sequences described in the application is the amino acid sequence of the α-1,3-fucosyltransferase Med1 (SEQ ID NO: 1) and the DNA sequences encoding the α-1,3-fucosyltransferase Med 1 (SEQ ID NO: 2), the DNA sequence encoding the colanic acid gene cluster from E. coli (SEQ ID NO: 3), the amino acid sequence of the lactose permease LacY from E. coli (SEQ ID NO: 6), the amino acid sequence of the β-1,3-N-acetylglucosaminyltransferase LgtA from N. meningitidis (SEQ ID NO: 4), the amino acid sequence of the β-1,4-galactosyltransferases galT from H. pylori (SEQ ID NO: 5), the amino acid sequence of the β-1,3-galactosyltransferases galTK from H. pylori (SEQ ID NO: 31), the amino acid sequence of the α-1,2-fucosyltransferase Smob from Sulfuriflexus mobilis (SEQ ID NO: 32), the amino acid sequence of the α-1,3-fucosyltransferase Para1 from Parabacteroides sp. AM08-6 (SEQ ID NO: 33), and the DNA sequence encoding the α-1,3-fucosyltransferase Para1 from Parabacteroides sp. AM08-6 (SEQ ID NO: 34), the amino acid sequence of the α-1,3-fucosyltransferase FutB from Helicobacter pylori (SEQ ID NO: 35), the nucleic acid sequence encoding the α-1,3-fucosyltransferase FutB from Helicobacter pylori (SEQ ID NO: 36), the amino acid sequence of the α-1,3-fucosyltransferase CafD from Helicobacter hepaticus ATCC 51449 (SEQ ID NO: 37), the amino acid sequence of the α-1,3-fucosyltransferase FucT109 from Bacteroides fragilis NCTC 9343 (SEQ ID NO: 38), the nucleic acid sequence encoding the α-1,3-fucosyltransferase CafD from Helicobacter hepaticus ATCC 51449 (SEQ ID NO: 39) and the nucleic acid sequence encoding the α-1,3-fucosyltransferase α-1,3-fucosyltransferase FucT109 from Bacteroides fragilis NCTC 9343 (SEQ ID NO: 40).

### Items

1. A genetically engineered cell capable of producing one or more fucosylated HMOs, comprising a recombinant nucleic acid sequence encoding an enzyme with α-1,3(4)-fucosyltransferase activity, wherein the enzyme is Med1 with an amino acid sequence according to SEQ ID NO: 1, or a functional homologue thereof with an amino acid sequence that is at least 80% identical to SEQ ID NO: 1.
2. The genetically engineered cell according to item 1, wherein the fucosylated HMOs is selected from complex fucosylated HMOs comprising at least five monosaccharide units.
3. The genetically engineered cell according to claim 1 or 2, wherein the fucosylated HMOs is/are selected from fucosylated HMOs with an LNT and/or LNnT backbone structure.
4. The genetically engineered cell according to item 1 or 2, wherein the fucosylated HMOs is, LNFP-III.
5. The genetically engineered cell according to item 1 or 2, wherein the fucosylated HMOs is LNFP-II and/or LNDFH-1
6. The genetically engineered according to items 1 to 5, wherein the α-1,3(4)-fucosyltransferase only fucosylates a N-acetylglucoseamine (GlcNAc) moiety in an acceptor oligosaccharide.
7. The genetically engineered according to item 6, wherein the acceptor oligosaccharide for the α-1,3(4)-fucosyltransferase has a LNT or LNnT backbone.
8. The genetically engineered according to item 6 or 7, wherein the acceptor oligosaccharide for the α-1,3(4)-fucosyltransferase is LNT or LNnT.
9. The genetically engineered cell according to any of the preceding items, wherein the α-1,3(4)-fucosyltransferase does not fucosylate glucose and galactose moieties in an acceptor oligosaccharide, in particular in an acceptor oligosaccharide with an LNT or LNnT backbone.
10. The genetically engineered cell according to any of the preceding items, wherein the genetically engineered cell comprises one or more further recombinant nucleic acids encoding one or more heterologous glycosyltransferases.
11. The genetically engineered cell according to any one of the preceding items, wherein the cell further comprises a recombinant nucleic acid sequence encoding a β-1,3-galactosyltransferase and/or a β-1,4-galactosyltransferase.
12. The genetically engineered cell according to item 10 or 11, wherein the genetically engineered cell further comprises a recombinant nucleic acid sequence encoding a β-1,3-N-acetyl-glucosaminyltransferase.
13. The genetically engineered cell according to any one of items 11 or 12, wherein the β-1,3-N-acetylglucosaminyltransferase is from Neisseria meningitidis and the β-1,3-galactosyltransferase is from Helicobacter pylori.
14. The genetically engineered cell according to item 10 to 13, wherein the 1,3-N-acetylglucosaminyltransferase has an amino acid sequence according to SEQ ID NO: 4, or a functional homologue thereof with an amino acid sequence that is at least 80% identical to SEQ ID NO: 4 and the β-1,3-galactosyltransferase is has an amino acid sequence according to SEQ ID NO: 31, or a functional homologue thereof with an amino acid sequence that is at least 80% identical to SEQ ID NO: 31.
15. The genetically engineered cell according to any one of items 11 to 13, wherein the 1,3-N-acetylglucosaminyltransferase has an amino acid sequence according to SEQ ID NO: 4, or a functional homologue thereof with an amino acid sequence that is at least 80% identical to SEQ ID NO: 4 and the β-1,4-galactosyltransferase is has an amino acid sequence according to SEQ ID NO: 5, or a functional homologue thereof with an amino acid sequence that is at least 80% identical to SEQ ID NO: 5.
16. The genetically engineered cell according to any of the preceding items, wherein the cell further comprises a recombinant nucleic acid sequence encoding an α-1,2-fucosyltransferase(s).
17. The genetically engineered cell according to item 16, wherein α-1,2-fucosyltransferase(s) is from Sulfuriflexus mobilis.
18. The genetically engineered cell according to item 15 or 10, wherein the α-1,2-fucosyltransferase has an amino acid sequence according to SEQ ID NO: 32, or a functional homologue thereof with an amino acid sequence that is at least 80% identical to SEQ ID NO: 32.
19. The genetically engineered cell according to any of the preceding items, wherein the recombinant nucleic acid sequences individually are under the control of a promoter selected from the group consisting of PglpF, Plac, PmglB_70UTR, PglpA_70UTR and PglpT_70UTR (SEQ ID NOs: 19, 28, 16, 17 and 18, respectively) and variants thereof.
20. The genetically engineered cell according to item 19, wherein the promoter is a strong promoter selected from the group consisting of SEQ ID NOs 7, 8, 9, 10, 11, 12, 13, 14, 15, 16, 17, 18 and 19.
21. The genetically engineered cell according to any of the preceding items, wherein the cell comprises one, two, three or more genomic copies of the recombinant nucleic acid sequence encoding the glycosyltransferase Med1 with an amino acid sequence according to SEQ ID NO: 1, or a functional homologue thereof with an amino acid sequence that is at least 80% identical to SEQ ID NO: 1.
22. The genetically engineered cell according to any of the preceding items, wherein the recombinant nucleic acid sequence encoding the glycosyltransferase Med1 with an amino acid sequence according to SEQ ID NO: 1, or a functional homologue thereof with an amino acid sequence that is at least 80% identical to SEQ ID NO: 1, is encoded on a plasmid.
23. The genetically engineered cell according to item 22, wherein the plasmid is high copy number plasmid.
24. The genetically engineered cell according to item 22 or item 23, wherein the plasmid is a pUC57 plasmid.
25. The genetically engineered cell according to any of the preceding items, wherein the cell comprises one, two, three or more genomic copies and a plasmid borne copy of the recombinant nucleic acid sequence encoding the glycosyltransferase Med1 with an amino acid sequence according to SEQ ID NO: 1, or a functional homologue thereof with an amino acid sequence that is at least 80% identical to SEQ ID NO: 1.
26. The genetically engineered cell according to any of the preceding items, wherein the cell overexpresses at least one enzyme in the de novo GDP-fucose pathway responsible for the formation of GDP-fucose.
27. The genetically engineered cell according to item 26, wherein the one or more enzyme is selected from the group consisting of mannose-6 phosphate isomerase (manA), phosphomannomutase (manB), mannose-1-phosphate guanylyltransferase guanylyltransferase (manC), GDP-mannose-4,6-dehydratase (gmd) and GDP-L-fucose synthase (wcaG).
28. The genetically engineered cell according to any of the preceding items, wherein the cell comprises a recombinant nucleic acid sequence according to SEQ ID NO: 3, encoding the colanic acid (CA) gene cluster.
29. The genetically engineered cell according to any of the preceding items, wherein said engineered cell is a microorganism.
30. The genetically engineered cell according to any of the preceding items, wherein said engineered cell is a bacterium or a fungus.
31. The genetically engineered cell according to item 30, wherein said fungus is selected from a yeast cell of the genera Komagataella, Kluyveromyces, Yarrowia, Pichia, Saccaromyces, Schizosaccharomyces or Hansenula or from a filamentous fungus of the genera Aspergillus, Fusarium or Thricoderma.
32. The genetically engineered cell according to item 30, wherein said bacterium is selected from the group consisting of Escherichia sp., Bacillus sp., lactobacillus sp., Corynebacterium sp. and Campylobacter sp.
33. The genetically engineered according to any of item 29 to 32 wherein said engineered cell is selected from the group consisting of Escherichia Coli, Bacillus subtilis, lactobacillus lactis, Corynebacterium glutamicum Yarrowia lipolytica, Pichia pastoris, and Saccharomyces cerevisiae.
34. The genetically engineered cell according to item 32 or 33, wherein said engineered cell is a microorganism is E. coli.
35. The genetically engineered cell according to any of the preceding items, wherein at least 5% of the molar content of the total HMOs produced by said cell are complex fucosylated HMOs.
36. The genetically engineered cell according to any of item 1 to 15 or 19 to 35, wherein at least 5% of the molar content of the total HMOs produced by said cell is LNFP-II.
37. The genetically engineered cell according to any of items 1 to 15 or 19 to 35, wherein at least 50% of the molar content of the total HMOs produced by said cell is LNFP-III.
38. The genetically engineered cell according to any of items 1 to 35, wherein at least 9% of the molar content of the total HMOs is LNDFH-I.
39. The genetically engineered cell according to any of the preceding items, wherein the cell produces a mixture of HMOs comprising LNFP-II and LNT or LNFP-III and LNnT, or LNFP-I and LNDFH-I.
40. The genetically engineered cell according to any of the preceding items, wherein the cell produces a mixture of HMOs essentially consisting of
   a) LNFP-II and LNT, or
   b) LNFP-II and 3FL, or
   c) LNFP-II, LNT and 3FL, or
   d) LNFP-II, LNT, 3FL and pLNH2, or
   e) LNFP-III and LNnT, or
   f) LNFP-III, LNnT and pLNnH, or
   g) LNFP-III and 3FL, or
   h) LNDFH-I, LNFP-I and 2′FL.
41. A method for producing one or more fucosylated HMOs, said method comprising culturing a genetically engineered cell according to any of items to 40.
42. The method according to item 41, wherein the method comprises cultivating the genetically engineered cell in the presence of an energy source selected from the group consisting of glucose, sucrose, fructose, xylose and glycerol.
43. The method according to any one of items 41 to 42, wherein lactose is added during the cultivation of the genetically engineered cells as a substrate for the HMO formation.
44. The method according to item 41 to 43, wherein the fucosylated HMOs is selected from the group consisting of LNFP-II, LNFP-III and LNDFH-I.
45. The method according to item 41 to 44, wherein the method produces a mixture of HMOs and wherein said mixture is optionally purified to at least 75% purity.
46. The method according to item 41 to 44, wherein the fucosylated HMO(s) produced by the method is purified to at least 75% purity.
47. The method according to item 41 to 46, wherein one of the fucosylated HMO is selected from the group consisting of LNFP-II, LNFP-III and LNDFH-I and is purified to at least 75% purity.
48. Use of an enzyme with α-1,3(4)-fucosyltransferase activity in production of a fucosylated product, wherein the enzyme is Med1 with an amino acid sequence according to SEQ ID NO: 1, or a functional homologue thereof with an amino acid sequence that is at least 80% identical to SEQ ID NO: 1.
49. The use of an enzyme according to item 48, wherein the fucosylated product is one or more fucosylated oligosaccharides.
50. The use of an enzyme according to any of items 48 or 49, wherein the fucosylated product is one or more fucosylated HMOs.
51. The use of an enzyme according to item 50, wherein the fucosylated product is LNFP-II or LNFP-III or LNDFH-I.
52. The use of an enzyme according to item 48, wherein the fucosylated product is one or more fucosylated polypeptides.
53. A mixture of HMOs consisting essentially of
   a) LNFP-II, pLNH2 and LNT, or
   b) LNFP-II, LNT, 3FL and pLNH2, or
   c) LNT-II, LNFP-II, LNT, 3FL and pLNH2, or
   d) LNFP-III and LNnT, or
   e) LNFP-III, LNnT and pLNnH, or
   f) LNFP-III and 3FL or
   g) LNDFH-I, LNFP-I and 2′FL.
54. The mixture of HMOs according to item 53, consisting essentially of 5-35% LNFP-II, 50-85% LNT, 0-15% LNT-II, 0-20% 3FL, and 0-5% pLNH2 with the total molar HMO content in the mixture adding up to 100%,
55. The mixture of HMOs according to item 53, consisting essentially of 50-90% LNFP-III, 0-40% LNnT, 0-20% 3FL and 0-8% pLNnH, with the total molar HMO content in the mixture adding up to 100%.
56. The mixture of HMOs according to item 53, consisting essentially of 65-75% LNFP-I and 7-15% LNDFH-I and 5-20% 2′FL with the total molar HMO content in the mixture adding up to 100%.
57. A composition comprising a mixture of HMOs, wherein the composition comprises 5-35% LNFP-II, 50-85% LNT, 0-15% LNT-II, 0-20% 3FL, and 0-5% pLNH2 of the total molar HMO content in the composition.
58. A composition comprising a mixture of HMOs, wherein the composition comprises 50-90% LNFP-III, 0-40% LNnT, 0-20% 3FL and 0-8% pLNnH of the total molar HMO content in the composition.
59. A composition comprising a mixture of HMOs, wherein the composition comprises 65-75% LNFP-I and 7-15% LNDFH-I and 5-20% 2′FL of the total molar HMO content in the composition.
60. Use of a mixture or composition according to any of items 53 to 59 as a dietary supplement or medical nutrition.
61. Use of a mixture or composition according to any of items 53 to 59 in infant nutrition.
62. A method for producing a fucosylated product, said method comprising,
   a. providing a purified fucosyltransferase, Med1, with an amino acid sequence according to SEQ ID NO: 1 or a functional homologue thereof with an amino acid sequence that is at least 80% identical to SEQ ID NO: 1,
   b. reacting the purified fucosyltransferase of a) with
      i. a substrate, such as an oligosaccharide or a glycosylated polypeptide, and
      ii. a fucose donor, such as GDP-fucose, and
   c. retrieving one or more fucosylated products from the reaction in b), and optionally purify said product.
63. The method according to item 62, wherein the fucosylated product is purified to at least 75% purity.

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Enzymes:

Screening of 50 enzymes with fucosyltransferase activity provided a single enzyme, Med1, which was capable of only fucosylating the GlcNAc moiety in both LNT and LNnT, meaning it possess very specific alpha-1,4-fucosyltransferase activity and alpha-1,3-fucosyltransferase activity (also written as alpha-1,3(4)-fucosyltransferase activity). One other enzyme, paral, with alpha-1,4-fucosyltransferase activity towards the GlcNAc moiety in LNT was identified. Para1 enzyme, however, also has alpha-1,3-fucosyltransferase activity towards the glucose moiety of LNT. Both enzymes therefore possess alpha-1,3(4)-fucosyltransferase activity and were capable of producing the complex HMO LNFP-II when introduced into a strain with LNT background. The GenBank ID and origin of the two fucosyltransferases Med1 and Para1, are provided in table 5 together with the three prior art enzymes, the additional 45 enzymes that were tested are not shown since they were not able to fucosylate the GlcNAc moiety of LNT.

TABLE 5
List of the enzymes for which results are shown
in the framework of the present invention

| Enzyme | | SEQ | |
|---|---|---|---|
| Name | GenBank ID* | ID NO | Origin |
| Med1 | WP_087337236.1 | 1 | Mediterranea sp. An20 |
| Para1 | WP_147381213.1 | 33 | Parabacteroides sp. AM08-6 |
| FutB** | WP_000487430.1 | 35 | Helicobacter pylori |
| CafD*** | AAP76669.1 | 37 | Helicobacter hepaticus |
| | | | ATCC 51449 |
| FucT109 | CAH09151.1 | 38 | Bacteroides fragilis |
| | | | ATCC 25285 |

*the sequences used in the present application may be truncated at the N- or C-terminal as compared to the GenBank sequence, these are represented by the SEQ ID NO.
**FutB is known to produce a mixture of LNFP-III and LNFP-VI (Dumon et al 2004 Biotechnol. Prog. 20: 412-419).
***CafD has been suggested to produce LNFP-III in WO2016/040531
\hat{ } FucT109 has been suggested to produce LNFP-III in WO 2019/008133

### Strains

The strains (genetically engineered cells) constructed in the present application were based on Escherichia coli K-12 DH1 with the genotype: F⁻, λ⁻, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene. Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

To obtain an LNT producing strain the MDO strain was further engineered by chromosomally integrating a beta-1,3-GlcNAc transferase (LgtA from Neisseria meningitidis, homologous to NCBI Accession nr. WP_033911473.1 and as shown in SEQ ID NO: 4) and a beta-1,3-galactosyltransferase (GalTK from Helicobacter pylori, homologous to GenBank Accession nr. BD182026.1 and as shown in SEQ ID NO: 31) both under the control of a PglpF promoter (SEQ ID NO: 19), this strain is named the LNT strain.

To obtain an LNnT producing strain the MDO strain was further engineered by chromosomally integrating a beta-1,3-GlcNAc transferase (LgtA from Neisseria meningitidis, homologous to NCBI Accession nr. WP_033911473.1 and shown as SEQ ID NO: 4) and a beta-1,4-galactosyltransferase (GalT from Helicobacter pylori, homologous to GenBank ID WP_001262061.1 and shown as SEQ ID NO: 5) both under the control of a PglpF promoter (SEQ ID NO: 19), this strain is named the LNnT strain.

Codon optimized DNA sequences encoding med1 or para1 were genomically integrated into the LNT strain. Furthermore, med1 was integrated into an LNnT strain and LNFP-I strain.

The genotypes of the background strain (MDO), the LNT strain, the LNnT strain and the α-1,3(4)-fucosyltransferase expressing strains capable of producing LNFP-II and/or LNDFH-I are provided in Table 6.

TABLE 6
Genotypes of the strains, capable of producing LNFP-II,
LNFP-III and/or LNDFH-I, used in the present examples.

| | | a1,3(4)FT* |
|---|---|---|
| | | cDNA SEQ |
| Strain ref | Genotype | ID NO |
| MDO | F− λ− ΔendA1 ΔrecA1 ΔrelA1 ΔgyrA96 Δthi-1 glnV44 | — |
| | hsdR17(rK−mK−) ΔlacZ wcaF::Plac ΔnanKETA ΔlacA | |
| | ΔmelA ΔwcaJ ΔmdoH | |
| LNT | MDO, 2x lgtA-PglpF¹, 1x galTK-PglpF², | — |
| LNnT | MDO, 2x lgtA-PglpF¹, 1x galT-PglpF³ | — |
| LNFP-I | MDO, 2x lgtA-PglpF¹, 1x galTK-PglpF², 1x Smob-PglpF⁷ | — |
| FutB_LNT | LNT, FutB-PglpF⁵, PglpF-CA⁴ | 36 |
| CafD_LNT | LNT, CafD-PglpF, CA-PglpF⁴ | 39 |
| FucT109_LNT | LNT, FucT109-PglpF⁵, PglpF-CA⁴ | 40 |
| Para1_1 | LNT, Para1-PglpF⁵, PglpF-CA⁴ | 34 |
| Para1_1 | LNT, 2x Para1-PglpF⁵, PglpF-CA⁴ | 34 |
| Med1_1 | LNT, 1x med1-PglpF⁶, PglpF-CA⁴ | 2 |
| Med1_2 | LNT, 2x med1-PglpF⁶, PglpF-CA⁴ | 2 |
| Med1_3 | LNT, 2x med1-PglpF⁶, pUC57-Med1-PglpF⁶, PglpF-CA⁴ | 2 |
| Med1_4 | LNFP-I, 2x Med1-PglpF⁶, PglpF-CA⁴ | 2 |
| Med1_5 | LNFP-I, 3x Med1-PglpF⁶, PglpF-CA⁴ | 2 |
| Med1_6 | LNnT, 1x med1-PglpF⁶, PglpF-CA⁴ | 2 |
| Med1_7 | LNnT, 2x med1-PglpF⁶, PglpF-CA⁴ | 2 |
| Med1_8 | LNnT, 2x med1-PglpF⁶, pUC57-Med1-PglpF⁶, PglpF-CA⁴ | 2 |
| FutB_LNnT | LNnT, FutB-PglpF, CA-PglpF⁴ | 36 |
| CafD_LNnT | LNnT, CafD-PglpF, CA-PglpF⁴ | 39 |
| FucT109_LNnT | LNnT, FucT109-PglpF, CA-PglpF⁴ | 40 |

*1,3(4)FT is an abbreviation of alpha-1,3(4)-fucosyltransferase
¹lgtA-PglpF - two genomically inserted copies of a gene encoding β-1,3-N-acetyl-glucosaminyltransferase (SEQ ID NO: 4) under control of a PglpF promoter (SEQ ID NO: 19).
²galTK-PglpF - one genomically inserted gene encoding β-1,3-Galactosyltransferase (SEQ ID NO: 31) under control of a PglpF promoter (SEQ ID NO: 19).
³galT-PglpF - one genomically inserted gene encoding β-1,4-Galactosyltransferase (SEQ ID NO: 5) under control of a PglpF promoter (SEQ ID NO: 19).
⁴CA = extra colanic acid gene cluster (gmd-wcaG-wcaH-wcaI-manC-manB, SEQ ID NO: 3) under the control of a PglpF promoter (SEQ ID NO: 19) at a locus that is different than the native locus.
⁵Para1-PglpF - one genomically inserted gene encoding α-1,3(4)-fucosyltransferase (SEQ ID NO: 33) under control of a PglpF promoter (SEQ ID NO: 19).
⁶med1-PglpF- gene encoding α-1,3(4)-fucosyltransferase (SEQ ID NO: 2) under control of a PglpF promoter (SEQ ID NO: 19). Either one (1x), two (2x), or three (3x) genomically inserted copies or expression from plasmid pUC57, which is high-copy number (>300) plasmid having the ColE1/pMB1/pBR322/pUC origin of replication. The antibiotic resistance marker on the pUC57 vector is ampicillin.
⁷Smob-PglpF one genomically inserted gene encoding α-1,2-fucosyltransferase (SEQ ID NO: 32) under control of a PglpF promoter.

### Deep Well Assay

Deep Well Assays in the current examples were performed as originally described to Lv et al (Bioprocess Biosyst Eng 20 (2016) 39:1737-1747) and optimized for the purposes of the current invention. More specifically, the strains disclosed in the present example were screened in 96 deep well plates using a 4-day protocol. During the first 24 hours, precultures were grown to high densities (OD600 up to 5) and subsequently transferred to a medium that allowed induction of gene expression and product formation.

More specifically, during day 1, fresh precultures were prepared using a basal minimal medium (BMM) (pH 7.0) supplemented with magnesium sulphate (0.12 g/L), thiamine (0.004 g/L) and glucose (5.5 g/L). Basal Minimal medium had the following composition: NaOH (1 g/L), KOH (2.5 g/L), KHzPO4 (7 g/L), NH&HzPO4 (7 g/L), Citric acid (0.5 g/I), trace mineral solution (5 mL/L). The trace mineral stock solution contained; ZnSO~*7H~O 0.82 g/L, Citric acid 20 g/L, Mn$04*H&O 0.98 g/L, FeSO4*7H&O 3.925 g/L, CuSO4*5H~O 0.2 g/L. The pH of the Basal Minimal Medium was adjusted to 7.0 with 5 N NaOH and autoclaved. The precultures were incubated for 24 hours at 34° C. and 1000 rpm shaking and then further transferred to 0.75 mL of a new BMM (pH 7.5) to start the main culture. The new BMM was supplemented with magnesium sulphate (0.12 g/L), thiamine (0.02 g/L), a bolus of glucose solution (0.1-0.15 g/L) and a bolus of lactose solution (5-20 g/L) Moreover, a 20% stock solution of sucrose (40-45 g/L) or maltodextrin (19-20 g/L) was provided as carbon source, accompanied by the addition of a specific hydrolytic enzyme, sucrose hydrolase or glycoamylase, respectively, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analysed by HPLC to identify the HMOs produced by the cell. pLNH2 is not officially reported as an HMO although it may very well be present in human mothers' milk in small amounts. In the present application it is included in the total HMO calculations.

### Fermentation—Ambr

The E. coli strains were cultivated in 250 mL fermenters (Ambr250 HT Bioreactor system, Sartorius) starting with 100 mL of mineral culture medium consisting of 30 g/L glucose and a mineral medium comprised of NH₄H₂PO₄, KH₂PO₄, MgSO₄×7H₂O, NaOH, citric acid, trace element solution, antifoam and thiamine. The dissolved oxygen level was kept at 20% by a cascade of first agitation and then airflow starting at 700 rpm (up to max 4500 rpm) and 1 VVM (up to max 3 VVM). The pH was kept at 6.8 by titration with 8.5% NH₄OH solution. The cultivations were started with 2% (v/v) inoculums from pre-cultures comprised of 10 g/L glucose, (NH₄)₂HPO4, KH₂PO₄, MgSO₄×7H₂O, KOH, NaOH, citric acid, trace element solution, antifoam and thiamine. After depletion of the glucose contained in the basal minimal medium, a glucose containing feed solution was continuously added to the fermenter at a rate that maintained carbon-limiting conditions. The temperature was initially at 33° C. but was dropped to 30° C. after 3 hours of feeding. Lactose was added as a bolus addition of 25% lactose monohydrate solution 36 hours after feed start and then every 19 hours to keep lactose from being a rate limiting factor. The growth, metabolic activity and metabolic state of the cells was followed by on-line measurements of reflectance and CO₂ evolution rate. Throughout the fermentations, samples were taken to determine the concentration of HMO products, lactose and other minor by-products using HPLC.

### Fermentation—Dasbox

The E. coli strains were cultivated in 100 mL fermenters (Dasbox, Eppendorf) starting with 100 mL of mineral culture medium consisting of 30 g/L glucose and a mineral medium comprised of NH₄H₂PO₄, KH₂PO₄, MgSO₄×7H₂O, KOH, NaOH, citric acid, trace element solution, antifoam and thiamine. The dissolved oxygen level was kept at 20% by a cascade of first agitation and then airflow starting at 700 rpm (up to max 2000 rpm) and 1 VVM (up to max 3 VVM). The pH was kept at 6.8 by titration with 8.5% NH₄OH solution. The cultivations were started with 2% (v/v) inoculums from pre-cultures comprised of 10 g/L glucose, (NH₄)₂HPO4, KH₂PO₄, MgSO₄×7H₂O, KOH, NaOH, citric acid, trace element solution, antifoam and thiamine. After depletion of the glucose contained in the basal minimal medium, a feed solution containing glucose, MgSO₄×7H₂O, H₃PO₄ and trace mineral solution was continuously added to the fermenter at a rate that maintained carbon-limiting conditions. The temperature was initially at 33° C. but was dropped to 25° C. initiated 15 min after the start of the feed. Lactose was added as bolus additions of 25% lactose monohydrate solution at feed start and also together with the glucose feed to keep lactose from becoming a rate limiting factor. The growth, metabolic activity and metabolic state of the cells was followed by on-line measurements of agitation, dissolved oxygen tension, reflectance, NH₄OH base addition, O₂ uptake rate and CO₂ evolution rate. Throughout the fermentations, samples were taken to determine the concentration of HMO products, lactose and other minor by-products using HPLC.

### Example 1—In Vivo LNFP-II Synthesis

In the present example genetically modified cells expressing individual fucosyltransferase enzymes were screened for their ability to produce the fucosylated HMO LNFP-II, in an LNT strain with an additional colonic acid gene cluster to secure sufficient GDP-fucose as donor material for the fucosyltransferase.

Using the deep well assay described in the “Method” section, only two of the tested enzymes, med1 and para1, possessed alpha-1,4-fucosyltransferase activity towards the GlcNAc moiety in LNT, which is necessary to produce LNFP-II (table 5).

The initial screening of the fucosyltransferases were done with a single genomic copy. In table 6 the genotype of these strains are shown with the strain references Para1_1 and Med1_1. Based on the results of the initial screening further strains with additional copy numbers of the respective fucosyltransferase were generated. table 6 lists the genotype of the strains capable of producing LNFP-II.

The molar content of individual HMOs produced by the strains was measured by HPLC. The results of the LNFP-II producing cells as well as the three prior art strains are shown in table 7 as the fraction of the total HMO content (in percentage, %) produced by each strain.

TABLE 7
Content of individual HMO's as % of total HMO (mM) content produced by each strain.

| | FT* gene | | | | | | | |
|---|---|---|---|---|---|---|---|---|
| | copy | | | | | | | |
| | number | LNT-II | 3FL | LNT | LNFP-II | LNFP-V | LNDFH-II | pLNH2 |
| FutB_LNT | 1 | 0 | 0 | 64 | 0 | 33 | 0 | 2 |
| CafD_LNT | 1 | 0 | 0 | 96 | 0 | 0 | 0 | 4 |
| FucT109_{—} | 1 | 0 | 3 | 39 | 0 | 57 | 0 | 0 |
| LNT | | | | | | | | |
| Para1_1 | 1 | 38 | 1 | 51 | 4 | 4 | 2 | 0 |
| Med1_1 | 1 | 15 | 2 | 77 | 5 | 0 | 0 | 1 |
| Para1_2 | 2 | 45 | 0 | 34 | 5 | 5 | 11 | 0 |
| Med1_2 | 2 | 0 | 0 | 81 | 15 | 0 | 0 | 4 |
| Med1_3 | >50 | 0 | 20 | 50 | 30 | 0 | 0 | 0 |

* FT = Fucosyltransferase
No additional HMOs beyond the ones indicated in table 7 were identified in the deep well assay.

From the data presented in table 7, it can be seen that strains with one copy Para1 and Med1 are both capable of producing LNFP-II in low amounts, where as the FutB and FucT109 strains only produce LNFP-V and the CafD strain does not fucosylated LNT at all.

For the Para1 and Med1 fucosyltransferases there is a distinct difference between the specificity, in that Med1 exclusively fucosylated LNT in the GlcNAc moiety with an alpha-1,4 linkage, thus only producing a single fucosylated HMO with an LNT backbone, specifically LNFP-II, whereas Para1 fucosylated LNT at the Glucose (Glc) moiety and the GlcNAc moiety, thus producing three fucosylated HMO with an LNT backbone, LNFP-II, LNFP-V and LNDFH-II, respectively.

Furthermore, increasing the copy number of Med1 and Para1 to two genetic copies increased the LNFP-II production by 10% from 5% to 15% for Med1, whereas Para1 only increased LNFP-II formation by 1%, whereas the amount of the difucosylated LNDFH-II was increased from 2 to 11%, clearly indicating that Para1 has equal preference for fucosylating the GlcNAc and the Glc moiety of LNT, whereas Med1 only possess activity towards the GlcNAc moiety. The capability to increase LNFP-II formation by further increasing the Med1 expression was investigated by introduction of the high copy plasmid pUC57-Med1-PglpF (Med1_3) containing the Med1 encoding sequence. The results in table 7 shows that this increase in Med1 expression increased the LNFP-II level from 15% to 30%. However, when Med1 was highly overexpressed, it appears that it is capable of fucosylating the Glc moiety on lactose to form 3FL.

Accordingly, the copy number variation may be used in the production to tailor specific HMOs mixtures comprising specific content of the individual HMOs, depending on the need for the specific product. Increasing the copy number of Med1 did not result in production of any additional fucosylated HMO species with an LNT backbone.

The absence of alternative fucosylated LNT species when using Med1 is highly advantageous and preferred if it is desired to purify LNFP-II from a broth which does not contain additional fucosylated HMOs of similar size such as LNFP-V or slightly larger such as LNDFH-II. Thus, when the aim is to produce high amounts of a single HMO, a high level of that specific HMO is beneficial.

### Example 2 In Vivo LNFP-III Synthesis

Considering the high specificity for the GlcNAc moiety of Med1 in example 1 it was investigated whether the same would apply with LNnT as the backbone, which would require alpha-1,3-fucosyltransferase activity (which was also observed in example 1 in high copy nr with lactose as substrate, where med1 produced some 3FL).

Genetically modified cells expressing Med1 were screened for their ability to produce the fucosylated HMO LNFP-III. The genotype of the Med1 strains generated for this example are shown in table 6) The strains were screened in a in a deep well assay setup as described in the “Method” section. The molar content of individual HMOs produced by the strains was measured by HPLC.

The results of the LNFP-III producing cells are shown in table 8 as the fraction of the total HMO content (in percentage, %) produced by each strain.

TABLE 8
Content of individual HMO's as % of total HMO (mM) content produced by each strain.

| | FT* copy | | | | | | |
|---|---|---|---|---|---|---|---|
| | number | LNFP-III | LNnT | pLNnH | 3FL | LNDFH-III | LNFP-VI |
| CafD_LNnT | 1 | 0 | 82 | 18 | 0 | 0 | 0 |
| FutB_LNnT | 1 | 6 | 79 | 13 | 1 | 0 | 1 |
| FucT109_LNnT | 1 | 26 | 29 | 1 | 2 | 24 | 18 |
| Med1_6 | 1 | 52 | 42 | 6 | 0 | 0 | 0 |
| Med1_7 | 2 | 90 | 10 | 0 | 0 | 0 | 0 |
| Med1_8 | >50 | 82 | 0 | 0 | 18 | 0 | 0 |

* FT: Fucosyltransferase
No additional HMOs beyond the ones indicated in table 8 were identified in the deep well assay.

From the data presented in table 8, it can be seen that Med1 can effectively transfer a fucosyl unit onto the GlcNAc moiety of LNnT in an alpha-1,3 linkage to produce high levels of LNFP-III. It can also be seen that Med1 does not produce any of the complex fucosylated HMOs having a fucosyl on the glc moiety (LNFP-VI and LNDFH-III), whereas FucT109 produce 18% LNFP-VI clearly indicating the FucT109 has similar specificity to both the GlcNAc and Glc moiety of LNnT. Likewise, FutB was found to produce minor amounts of LNFP-VI as well as more 3-FL with a single copy of the enzyme, indicating the FutB also have some activity to the terminal glucose (Glc) moiety of LNnT.

The prior art enzymes FutB and FucT109 only produced 6% and 26% LNFP-III of the total HMO, respectively. CafD which was purported to produce LNFP-III in WO2016/040531 appears to be unable to fucosylate LNnT or lactose in the present assay, as it does not produce any fucosylated HMOs.

Furthermore, Increasing the copy number of Med1 to two genetic copies (stain Med1_7) increased the LNFP-III production from 52% to 90%. With introduction of a high copy plasmid (pUC57-Med1-PglpF-amp) encoding Med1 (Strain Med1_8) a slight decrease in LNFP-III level down to 82% was observed and 18% 3FL was produced instead. Given a high copy number for the Med1 gene (strain Med1_8), all the LNnT produced by the cell was fucosylated, hence the formation of LNnT by the cell became the rate limiting step in the production of LNFP-III, potentially causing Med1 to fucosylate lactose instead of LNnT, thereby further decreasing the LNnT formation by consuming the lactose, leading to the decrease in LNFP-III.

Accordingly, the copy number variation may be used in the production to tailor specific HMO mixtures, in this case a mixture comprising LNFP-III, LNnT and/or 3FL in different ratios, depending on the need for the specific product. The data also indicate that if LNFP-III levels are to be increased further the copy number of the enzymes forming LNnT should potentially be increased together with the Med1 enzyme.

The absence of alternative similar sized fucosylated LNnT species (e.g. LNFP-VI or LNDFH-III) when using Med1 is highly advantageous and preferred if it is desired to produce pure LNFP-III.

### Example 3 In Vivo LNDFH-I Synthesis

In view of the results in example 1, where Med1 only showed activity towards the GlcNAc moiety of LNT, it was investigated whether Med1 would be able to produce the complex difucosylated hexasaccharide HMO LNDFH-I using an LNFP-I background strain with the alpha-1,2-fucosyltransferase, Smob in addition to two copies of the Med1 alpha-1,3(4) fucosyltransferase (Med1_4, table 6).

The strain was screened in the deep well assay setup as described in the “Method” section. The molar content of individual HMOs produced by the strains was measured by HPLC.

The results of the LNDFH-I producing cells are shown in table 9 as the fraction of the total HMO content (in percentage, %) produced by the Med1_4 strain.

TABLE 9
Content of individual HMO's as % of total
HMO (mM) content produced by each strain.

| | FT* copy number | 2′FL | 3FL | LNFP-I | LNDFH-I |
|---|---|---|---|---|---|
| Med1_4 | 2x Med1, | 17 | 0 | 70 | 12 |

*FT: Fucosyltransferase
No additional HMOs beyond the ones indicated in table 9 was identified in the deep well assay.

From the data presented in table 9, it can be seen that Med1 can transfer a fucosyl unit onto the GlcNAc moiety of LNFP-I in an alpha-1,4 linkage to form LNDFH-I. Production of difucosylated HMOs which require the presence of two fucosyltransferases with different activities, in this case alpha-1,2- and alpha 1,4-fucosyltransferase activity, are complex as it requires highly specific fucosyltransferases to produce the correct product without a lot of fucosylated side products. Steric hindrance may also be an issue related to the synthesis of complex HMOs, in the present case, the presence of the fucosyl moiety on the terminal galactose (Gal) unit of the LNT, could easily have caused a steric hindrance for the Med1 alpha1,4-fucosyltransferase or the fucosyl moiety on the GlcNAc could have caused steric hinderance for the Smob alpha1,2-fucosyltransferase. The 2′FL produced in the current strain results from the alpha-1,2-fucosyltransferase activity of the Smob enzyme, which is also capable of fucosylating lactose, which the Med1 alpha-1,3-fucosyltransferase activity apparently does not do in the present example, since no 3FL was detected, which confirms the observation in example 1 for the Med1 strain with two copies where no 3FL is produced (Med1_2 table 7).

### Example 4—Fermentation Using Med1 α-1,3(4)-Fucosyltransferase Strain for LNFP-II Production

To confirm the capability of producing LNFP-II without any complex fucosylated HMO side products as was observed in the deep well assays in example 1, the high copy number strain (Med1_3 strain) was fermented using the ambr fermentation as described in the “Method” section above. The results are shown in table 10.

TABLE 10
Content of individual HMO's as % of
total HMO content produced by the strain

| Strain ref. | FT copy number | 3FL | LNFP-II | LNT | pLNH2 |
|---|---|---|---|---|---|
| Med1_3 | >50 | 4 | 15 | 78 | 2 |

From the data presented table 10, it can be seen that the fraction of LNFP-II for the Med1_3 strain was higher when the culturing was done in deep well assays presented in example 1 compared to the fermentation, nevertheless showing the ability of Med1 expressing cells to produce only a single complex fucosylated HMO with an LNT backbone at a level of 15% of the total HMO produced by this strain. Interestingly, the 3FL level was also lower in the fermentation than what was observed in the deep-well assay and minor pLNH2 production was also observed.

### Example 5—Fermentation Using Med1 α-1,3(4)-Fucosyltransferase Strain for LNDFH-I Production

To confirm the capability of producing LNDFH-I as observed in the deep well assays in example 3, a strain containing 3× genomic copies of Med1-1,3(4)-fucosyltransferase and a single genomic copy of the Smob α-1,2-fucosyltransferase (Strain Med1_5, table 6) was generated. The strain was fermented using the ambr fermentation as described in the “Method” section above. The results are shown in table 11.

TABLE 11
Content of individual HMO's as % of
total HMO content produced by the strain

| Strain ref. | 2′-FL | LNFP-I | LNDFH-I |
|---|---|---|---|
| Med1_5 | 6 | 86 | 9 |

From the data presented table 11, it can be seen that the production of LNDFH-I in a fermentation setup could be confirmed using the Med1_5 strain, although the LNDFH-I level was slightly lower compared to the deep well assays presented in example 3.

### Example 6—Fermentation Using Med1 α-1,3(4)-Fucosyltransferase Strain for LNFP-III Production

To confirm the capability of producing LNFP-III as observed in the deep well assays in example 2, a strain containing 1× genomic copies of Med1-1,3(4)-fucosyltransferase (Strain Med1_6, table 6) was generated. The strain was fermented using the dashbox fermentation as described in the “Method” section above. The results are shown in table 12.

TABLE 12
Content of individual HMO's as % of
total HMO content produced by the strain

| Strain ref. | 3-FL | LNT-II | LNnT | LNFP-III | PLNnH | LNDFH-IIII |
|---|---|---|---|---|---|---|
| Med1_6 | 0 | 3 | 61 | 24 | 12 | 0 |

From the data presented table 12, it can be seen that using the using the Med1_6 strain in a fermentation setup the only complex fucosylated HMO produced is LNFP-III. The conversion from LNnT to LNFP-III was somewhat lower compared to the deep well assays presented in example 2, something which may be optimized, e.g., by using a strain with multiple Med1 copies on the genome.

### Example 7—Regeneration and Viability of Lyophilized Lactobacillus Species

Probiotics may be consumed as live bacteria or as a dried (e.g. lyophilized) product. Independent of the drying method, rehydration involves an important step in the recovery of dehydrated bacteria; an inadequate rehydration/regeneration step may lead to poor cell viability and a low final survival rate. Rehydration is therefore a highly critical step in the revitalization of a lyophilized culture. For both live and rehydrated bacteria, the survival of the bacteria under acidic conditions is critical since they need to pass through the acidic environment of the stomach and may also be faced with storage (shelf-life) in acidic food products.

In the present example it was tested whether the mixture of HMOs produced by the LNDFH-I producing strain in example 3 and example 5 can provide a benefit in the rehydration (regeneration) and viability of the probiotics. The test was performed under acidic conditions to resemble the conditions bacteria have to survive when passing through the stomach or when dosed in an acidic beverage.

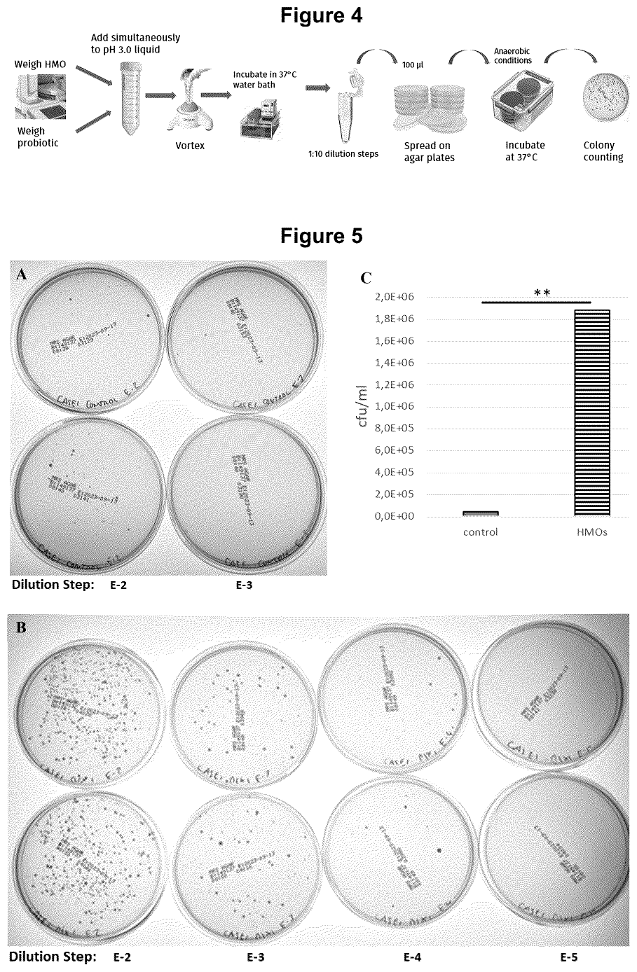

The lyophilized probiotics Lactobacillus casei (DSM 32382) (0.4 mg/ml) alone (control) or in combination with a mixture of HMOs consisting of 75% LNFP-I, 10% LNDFH-I and 15% 2′FL dosed in 5% w/v, were dissolved in sterile phosphate-buffered saline (PBS, pH=3), warmed to 37° C. and vigorously mixed for about 30 sec until no visible clumps remained. The tubes were incubated at 37° C. for 3 h. The samples were further diluted and 100 μl were spread in duplicates onto MRS agar plates which were incubated for 48 h at 37° C. in anaerobic chambers. The regeneration and viability of the probiotics were determined by counting the colonies on the plates after 48 h of incubation, in case of slow colony growth incubation was extended to 72 h. For the experimental setup, see FIG. 4. Results are expressed as mean values with standard deviation of colony-forming units (CFU) per millilitre calculated from the colonies formed on the agar plate.

Lyophilized Lactobacillus casei with the HMO mixture according to the invention showed a strongly significant (P<0.01) enhanced regeneration and survivability compared to the control without the HMO mixture after 3 h incubation at 37° C. in pH 3.0 acidic condition as assessed by colony counting. The data are shown in FIG. 5C as cfu/ml. The plates forming the basis for the count are shown in FIGS. 5A (control) and 5B (HMO mix). These data clearly show that the regeneration and viability of Lactobacillus casei after exposure to low pH conditions, such as in the stomach or in an acidic beverage, can be improved in the presence of the mixture of HMOs.

To our knowledge it has not previously been shown that a mixture of LNFP-I, LNnT and 2′FL has the benefit of improving the regeneration and survivability of probiotics in an acidic environment.

1. A genetically engineered cell capable of producing one or more fucosylated human milk oligosaccharides (HMOs), comprising a recombinant nucleic acid sequence encoding a fucosyltransferase with α-1,3(4)-fucosyltransferase activity, wherein the fucosyltransferase is Med1 with an amino acid sequence according to SEQ ID NO: 1, or a functional homologue thereof with an amino acid sequence that is at least 80% identical to SEQ ID NO: 1.

2. The genetically engineered cell according to claim 1, wherein the one or more fucosylated HMOs comprise a fucosylated HMO comprising at least five monosaccharide units.

3. The genetically engineered cell according to claim 1, wherein the fucosyltransferase only fucosylates the N-acetylglucoseamine (GlcNAc) moiety of LNT, LNnT or LNFP-I.

4. The genetically engineered cell according to claim 1, wherein the one or more fucosylated HMOs are selected from the group consisting of LNFP-II, LNFP-III and LNDFH-I.

5. The genetically engineered cell according to claim 1, wherein the cell further comprises a recombinant nucleic acid sequence encoding a β-1,3-galactosyltransferase or a β-1,4-galactosyltransferase.

6. The genetically engineered cell according to claim 5, wherein the cell further comprises a recombinant nucleic acid sequence encoding a β-1,3-N-acetyl-glucosaminyltransferase.

7. The genetically engineered cell according to claim 5, wherein the β-1,3-N-acetyl-glucosaminyltransferase is from Neisseria meningitidis.

8. The genetically engineered cell according to claim 1, wherein the cell further comprises a recombinant nucleic acid sequence encoding an α-1,2-fucosyltransferase from Sulfuriflexus mobilis.

9. The genetically engineered cell according to claim 1, wherein the cell comprises two or more genomic or plasmid-borne copies of the recombinant nucleic acid sequence.

10. The genetically engineered cell according to claim 1, wherein said engineered cell is selected from the group consisting of Escherichia Coli, Bacillus subtilis, lactobacillus lactis, Corynebacterium glutamicum, Yarrowia lipolytica, Pichia pastoris, and Saccharomyces cerevisiae.

11. The genetically engineered cell according to any of the preceding claims, wherein the cell, in fermentative conditions, produces a mixture of HMOs consisting essentially of
a. LNFP-II, pLNH2 and LNT,
b. LNFP-II, LNT and 3FL,
c. LNFP-II, LNT, 3FL and pLNH2,
d. LNT-II, LNFP-II, LNT, 3FL and pLNH2,
e. LNFP-III and LNnT,
f. LNFP-III, LNnT and pLNnH,
g. LNFP-III and 3FL, or
h. LNDFH-I, LNFP-I and 2′FL.

12. A method for producing one or more fucosylated HMOs, said method comprising the following steps
a. providing a genetically modified cell according to claim 1; and
b. culturing the cell according to (a) in a suitable cell culture medium to produce the one or more fucosylated HMOs, and
c. optionally, purifying the one or more fucosylated HMOs.

13. The method according to claim 12, wherein
at least 5% of the molar content of the total HMOs produced by said cell is LNFP-II.

14.-15. (canceled)

16. A mixture of HMOs consisting essentially of
a. LNFP-II, pLNH2 and LNT,
b. LNFP-II, LNT, 3FL and pLNH2,
c. LNT-II, LNFP-II, LNT, 3FL and pLNH2,
d. LNFP-III and LNnT,
e. LNFP-III, LNnT and pLNnH,
f. LNFP-III and 3FL, or
g. LNDFH-I, LNFP-I and 2′FL.

17. (canceled)

18. The genetically engineered cell according to claim 5, wherein the β-1,3-galactosyltransferase is from Helicobacter pylori.

19. The genetically engineered cell according to claim 5, wherein the β-1,4-galactosyltransferase is from Helicobacter pylori.

20. The method according to claim 12, wherein at least 50% of the molar content of the total HMOs produced by said cell is LNFP-III

21. The method according to claim 12, wherein at least 9% of the molar content of the total HMOs produced by said cell is LNDFH-I.

22. The genetically engineered cell according to claim 1, wherein the cell further comprises recombinant nucleic acid sequences encoding a β-1,3-N-acetyl-glucosaminyltransferase and a galactosyltransferase selected from a β-1,3-galactosyltransferase or a β-1,4-galactosyltransferase.


SEQUENCE LISTING (40 sequences)

SEQ ID NO: 1 - protein, 302 aa, Mediterranea sp.
MRKILILFYNKLWGVSWENEACASIPDGFELTDDRSRMAEADAVVFHLPDLARCMDGEEIEKRDGQLWVTWNLECDDNYPWVEAPEIRDLFDLRIGYRQQDDVWYPYFSFIRAEAFTRLPPRRPPLDKACLFVSSPFNRSHRFEFLRELMRHTEVDSFGRMLHNKDLPADEGRKTLMEKIADYKFVIGVENALARDYVTEKFFQPLLAGTVPVYRGAPNVEEFAPGRHCFVDARAFRSPADLAGFMNRCYEHPALYDELTAWRTQPLQPGFVQKLEQVKEHPFVRLCRVVEEKIRRIEYKIT

SEQ ID NO: 2 - DNA, 909 bp, synthetic construct
  source 1..909: mol_type = other DNA
atgcgcaaaatcctgatcctgttctataacaaactgtggggtgttagctgggaaaatgaagcatgtgcaagcattccggatggttttgaactgaccgatgatcgtagccgtatggccgaagcagatgcagttgtttttcatctgccggatctggcacgttgtatggatggtgaagaaattgaaaaacgtgatggtcagctgtgggttacctggaatctggaatgtgatgataactatccgtgggttgaagcaccggaaattcgtgacctgtttgatctgcgtattggttatcgtcagcaggatgatgtttggtatccgtattttagctttattcgtgccgaagcgtttacccgtctgcctccgcgtcgtccgcctctggataaagcatgtctgtttgttagcagcccgtttaatcgtagccatcgttttgaatttctgcgtgaactgatgcgtcataccgaagttgatagctttggtcgtatgctgcataataaagatctgcctgcagatgaaggtcgtaaaaccctgatggaaaagattgccgattataagtttgtgatcggtgttgaaaatgcactggcacgtgattatgttaccgaaaaattctttcagccgctgctggcaggcaccgttccggtttatcgtggtgcaccgaatgttgaagaatttgcaccgggtcgtcattgttttgttgatgcacgtgcctttcgttcaccggcagatctggcaggctttatgaatcgttgttatgaacatccggcactgtatgatgaactgacagcatggcgtacccagcctctgcagcctggttttgttcagaaactggaacaggttaaagaacatccgtttgttcgtctgtgtcgtgttgttgaagagaaaattcgtcgcatcgagtacaaaatcacctaa

SEQ ID NO: 3 - DNA, 6706 bp, synthetic construct
  source 1..6706: mol_type = other DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 4 - protein, 332 aa, Neisseria meningitidis
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 5 - protein, 273 aa, Helicobacter pylori
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 6 - protein, 417 aa, Escherichia coli [sequence omitted]

SEQ ID NO: 7 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 8 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 9 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 10 - DNA, 141 bp, synthetic construct
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 11 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 12 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 13 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 14 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 15 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 16 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 17 - DNA, 189 bp, synthetic construct
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 18 - DNA, 239 bp, synthetic construct
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 19 - DNA, 300 bp, Escherichia coli
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 20 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 21 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 22 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 23 - DNA, 350 bp, synthetic construct
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 24 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 25 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 26 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 27 - DNA, 189 bp, synthetic construct; identical to SEQ ID NO: 17
  source 1..189: mol_type = other DNA

SEQ ID NO: 28 - DNA, 107 bp, Escherichia coli
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 29 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 30 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 31 - protein, 439 aa, Helicobacter pylori
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 32 - protein, 292 aa, Sulfuriflexus mobilis
MIISQIIGGLGNQMFQYAAGRALSLVRGQPLLLDVTGFAGYGLHQGFELQRVFDCPIGIATEEDVRGILGWQFSAGIRRIVARPGMAAFRRKGFIVEPHFHYWPEIKNVPRDCYLLGYWQSERYFRAATADIRADFSFKSPLVNRNAETAAQIDQVNAISLHMRRGDYVNNPKTSATHGLCSLDYYQAAIKFVSERVEEPFFFIFSDDIAWVKANLKLDFPCQYVDHNHGAESFNDMHLMSLCQHHIIANSSFSWWGAWLNSDPKKIVLAPKKWFANKNNIKDLFPPGWVSL

SEQ ID NO: 33 - protein, 314 aa, Parabacteroides sp.
MKKIKINFVDFWDGFNCFDNYFVNILTCYYDVEIDAKPDYLFYSVFGDNHLEYKNCIKIYYTGENDFPDFNFCDYAIGFQYFTFEDRSFRYPEYLLYEGYNKLMDKEKTIDKLLSNRKFCNFVYSNSKYAIPLREKFFHQLSKYKKVDSGGRLLNNVGGPVQNKLDFIKDYKFTIAFENSSLSGYTTEKLMEPMVVNSLPIYWGNPNVHLDFNVASIINLQSFSSFEEAIEEIVRLDNDDDAYLQKLSQPWLTQDQAEKNWEKELALFLCNIFDQPIEKARRRTDFGFGVMKKFREARFSRFLSYSIFRRLLNF

SEQ ID NO: 34 - DNA, 945 bp, synthetic construct
  source 1..945: mol_type = other DNA
atgaagaagatcaagatcaacttcgtggatttttgggatggctttaactgcttcgataactactttgtgaatatcctgacctgctattacgatgtggaaattgatgcaaaaccggactacctgttttatagcgtgtttggtgataaccacctggaatacaaaaactgcatcaagatctattacaccggtgaaaacgatttcccggattttaacttttgcgattatgccatcggcttccagtattttacctttgaagatcgtagctttcgctatccggaatatctgctgtatgaaggctataacaaactgatggacaaagagaaaaccatcgataaactgctgagcaaccgcaaattttgcaactttgtttacagcaacagcaaatatgcaattccgctgcgcgaaaaattctttcatcagctgagcaaatacaagaaagttgatagcggtggtcgtctgctgaataatgttggtggtccggttcagaataaactggatttcatcaaagactataaattcaccattgccttcgaaaatagcagcctgagcggttataccaccgaaaagctgatggaaccgatggttgttaatagcctgccgatttattggggtaatccgaatgttcacctggattttaatgttgccagcattattaacctgcagagctttagcagctttgaagaagccattgaagaaattgtgcgcctggataatgatgatgatgcatatctgcagaaactgagccagccgtggctgacccaggatcaggccgagaaaaattgggaaaaagaactggcactgtttctgtgcaacatttttgatcagccgattgaaaaagcacgtcgtcgtaccgattttggttttggtgttatgaaaaagtttcgcgaagcacgttttagccgttttctgagctatagcatttttcgtcgcctgctgaacttttaa

SEQ ID NO: 35 - protein, 476 aa, Helicobacter pylori
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNYDDLRVNYDDLRVNYDDLRVNYDDLRVNYDDLRVNYDDLRVNYDRLLQNASPLLELSQNTTFKIYRKAYQKSLPLLRTIRRWVKK

SEQ ID NO: 36 - DNA, 1431 bp, synthetic construct
  source 1..1431: mol_type = other DNA
atgtttcagccgctgctggatgcatttattgaaagcgcaagcattgagaaaatggtgagcaaaagccctccgcctccgctgaaaattgcagttgcaaattggtggggtgatgaagaaatcaaagagtttaaaaagagcgtcctgtactttattctgagccagcgttatgcaattaccctgcatcagaatccgaatgaaagcagcgatctggtttttagcaatccgctgggtgcagcacgtaaaattctgagctatcagaataccaaacgcgtgttttataccggtgaaaatgaaagcccgaacttcaacctgtttgattatgccattggtttcgatgagctggattttaatgatcgttatctgcgtatgccgctgtattatgcacatctgcattatgaagcagaactggttaatgataccaccgcaccgtataaactgaaagataatagcctgtatgcactgaaaaagccgagccatcattttaaagaaaatcatccgaatctgtgtgccgtggtgaatgatgaaagcgatctgctgaaacgtggttttgcaagctttgttgcaagcaatgcaaatgcaccgatgcgtaatgcattttatgatgccctgaatagcattgaaccggttaccggtggtggtagcgttcgtaataccctgggttataaagttggcaacaaaagcgaattcctgagccagtataaattcaatctgtgctttgaaaacagccaaggctatggttatgtgaccgagaaaattctggatgcctatttcagccataccattccgatttattggggtagcccgagcgttgcaaaagatttcaatccgaaaagctttgtgaacgtgcacgatttcaacaactttgatgaagccatcgactacatcaaatatctgcatacccatccgaatgcctatctggatatgctgtatgaaaatccgctgaatacactggatggtaaagcctatttctatcaggatctgagcttcaaaaagatcctggactttttcaaaaccatcctggaaaacgataccatctatcacaataacccgttcatcttttatcgcgatctgcacgaaccgctgattagtattgatgatctgcgtgtgaattatgatgacctgcgcgttaactacgatgatttacgcgttaattatgatgatcttcgggtcaactatgatgacttaagagtgaactacgacgatctgagagtcaattatgatgatttgagagttaattatgatgacttgcgtgtgaattatgatgatttgcgggtgaactacgacgacctgcgtgttaactatgatcgtctgctgcagaatgcaagccctctgctggaactgagccagaatacaacctttaaaatctatcgcaaggcctaccagaaaagcctgcctctgctgcgtaccattcgtcgttgggttaaaaagtaa

SEQ ID NO: 37 - protein, 320 aa, Helicobacter hepaticus
MKDDLVILHPDGGIASQIAFVALGLAFEQKGAKVKYDLSWFAEGAKGFWNPSNGYDKVYDITWDISKAFPALHIEIANEEEIERYKSKYLIDNDRVIDYAPPLYCYGYKGRIFHYLYAPFFAQSFAPKEAQDSHTPFAALLQEIESSPSPCGVHIRRGDLSQPHIVYGNPTSNEYFAKSIELMCLLHPQSSFYLFSDDLAFVKEQIVPLLKGKTYRICDVNNPSQGYLDLYLLSRCRNIIGSQGSMGEFAKVLSPHNPLLITPRYRNIFKEVENVMCVNWGESVQHPPLVCSAPPPLVSQLKRNAPLNSRLYKEKDNASA

SEQ ID NO: 38 - protein, 331 aa, Bacteroides fragilis
MCDCLSIILLVKMKKIYLKFVDFWDGFDTISNFIVDALSIQYEVVLSNEPDYLFYSCFGTSHLEYDCIKIMFIGENIVPDFNVCDYAIGFNYIDFGDRYLRLPLYAIYDGFSNLQNKKIDVNKALDRKFCSIVVSNNKWADPIRETFFKLLSSYKKVDSGGRAWNNIGGPVDNKLDFISQYKFNIAFENSRVLGYTTEKIMEPMQVNSIPVYWGNPLVGKDFNVDSFVNAHDFDSLERLVEYIIELDSSKDKYLEMLEKPWLLDKTYLDWKQLLLNFINNIMMKSYKDAKYLVNYGHAGKYRNEQRFWGRCERKFKLQRIIEYYSQLFDRK

SEQ ID NO: 39 - DNA, 963 bp, synthetic construct
  source 1..963: mol_type = other DNA
atgaaagatgatctggttatcctgcatcctgatggtggtattgcaagccagattgcatttgttgcactgggtttagcatttgaacagaaaggtgccaaagtgaaatatgacctgagctggtttgcagaaggtgcaaaaggtttttggaatccgagcaatggttatgacaaagtgtatgatattacctgggatatcagcaaagcatttccggcactgcatattgaaattgccaacgaagaagaaatcgagcgctacaaaagcaaatatctgattgataacgatcgcgtgattgattatgcaccgcctctgtattgttatggttataaaggtcgcatcttccactatctgtatgcaccgtttttcgcacagagctttgcaccgaaagaagcacaggatagtcataccccgtttgcagccctgctgcaagaaattgaaagcagcccgagtccgtgtggtgttcatattcgtcgtggtgatctgagccagccgcatattgtttatggtaatccgaccagcaatgagtattttgccaaaagcattgaactgatgtgtctgctgcatccgcagagcagcttttacctgtttagcgacgatctggcatttgtgaaagagcagattgttccgctgctgaaaggtaaaacctatcgtatttgtgatgtgaataatccgagtcagggttatctggatctgtatctgctgagccgttgtcgtaacattattggtagccagggtagcatgggtgaatttgcaaaagttctgagtccgcataatcctctgctgattacaccgcgttatcgcaacattttcaaagaagtggaaaatgtgatgtgcgtgaattggggtgaaagcgttcagcatccgcctctggtgtgtagcgcaccgccaccgctggttagccagctgaaacgtaatgcaccgctgaatagccgtctgtataaagaaaaagataatgccagcgcctaa

SEQ ID NO: 40 - DNA, 996 bp, synthetic construct
  source 1..996: mol_type = other DNA
atgtgtgattgcctgagcattattctgctggtgaaaatgaaaaagatctatctgaagttcgtggatttctgggatggctttgataccattagcaactttattgttgatgccctgagcatccagtatgaagttgttctgagcaatgaaccggattacctgttttatagctgttttggcaccagccatctggaatatgattgcatcaaaatcatgttcatcggcgaaaatatcgtgccggattttaatgtttgcgattatgccatcggcttcaactatattgattttggtgatcgttatctgcgtctgccgctgtatgcaatttatgatggttttagcaatctgcagaacaaaaagatcgatgtgaataaagccctggatcgcaaattttgcagcattgttgtttccaacaacaaatgggcagatccgattcgtgaaacctttttcaaactgctgagcagctataagaaagttgatagcggtggtcgtgcatggaataacattggtggtccggttgataataaactggatttcatcagccagtacaaatttaacattgcctttgaaaatagccgtgtgctgggttataccaccgagaaaatcatggaaccgatgcaggttaatagcattccggtttattggggtaatccgctggttggcaaagatttcaatgttgatagctttgtgaacgcccacgattttgatagcctggaacgtctggttgaatatatcattgaactggatagcagcaaggacaagtatctggaaatgctggaaaaaccgtggctgctggataaaacatatctggattggaaacagctgctgctgaactttattaacaacattatgatgaaaagctataaagatgccaaatatctggtgaactatggccatgcaggtaaatatcgtaatgaacagcgtttttggggtcgttgtgaacgtaaattcaagctgcagcgtatcatcgagtattacagccagctgtttgatcgcaagtaa